(19) United States
(12) Patent Application Publication
(10) Pub. No.: US 2008/0051585 A1
(43) Pub. Date: Feb. 28, 2008

(54) Process for the preparation of indolin-2-one derivatives useful as PR modulators

(75) Inventors: Alexander V. Gontcharov, Rivervale, NJ (US); John R. Potoski, West Nyack, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/893,335

(22) Filed: Aug. 15, 2007

Related U.S. Application Data
(60) Provisional application No. 60/838,447, filed on Aug. 17, 2006.

(51) Int. Cl. C07D 209/04

(52) U.S. Cl. 548/465; 548/512

(57) ABSTRACT

Processes for dialkylating indolinones, specifically indolin-2-ones are provided. The processes for dialkylating an indolin-2-one include performing the dialkylation in the presence of at least 2 equivalents of a first base, a second base containing at least 1 equivalent of lithium diisopropylamide, and an alkylating agent.

Processes for preparing a compound of the structure are provided, wherein R¹, R³, R⁴, R⁶, R⁷, R⁹, and R¹⁰ are as defined herein. 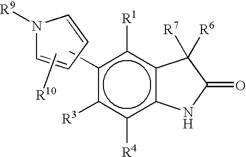 In one embodiment, a process for preparing 5-(4′-fluoro-2′-oxospiro[cyclopropane-1,3′-indoline]-5′-yl)-1-methyl-1H-pyrrole-2-carbonitrile is provided.

### CROSS-REFERENCE TO RELATED APPLICATIONS

This application claims the benefit of the priority of U.S. Provisional Patent Application No. 60/838,447, filed Aug. 17, 2006.

### BACKGROUND OF THE INVENTION

5-(4′-fluoro-2′-oxospiro[cyclopropane-1,3′-indoline]-5′-yl)-1-methyl-1H-pyrrole-2-carbonitrile is described in US Patent Application Publication No. US-2006/0030717, which is hereby incorporated by reference, and is a progesterone receptor modulator.

The process for preparing 5-(4′-fluoro-2′-oxospiro[cyclopropane-1,3′-indoline]-5′-yl)-1-methyl-1H-pyrrole-2-carbonitrile as described in US Patent Application Publication No. 2006/0030717 includes oxidation of the amine group of an aniline to a nitro group, conversion of the nitrobenzene to a dimalonate, reduction of the nitro group to an amine group, acid hydrolysis, decarboxylation, cyclization to the indolin-2-one, cyclopropanation at the 3-position, bromination of the cyclopropane indolinone intermediate, and Suzuki coupling of the bromide with a cyanopyrroleboronic acid. This route has some disadvantages, especially upon scale-up, including undesirable by-products, lower than optimal yields in the cyclopropanation step, and lower than optimal yields for the Suzuki coupling. See, Scheme 1.

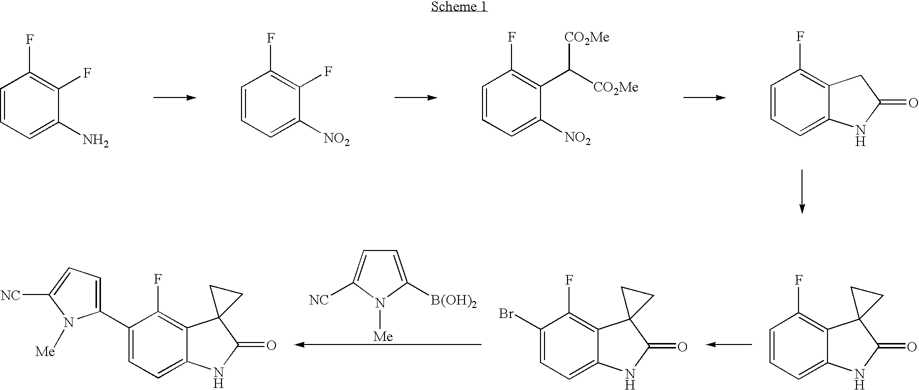

What is needed in the art are alternate processes for preparing PR modulators including indolinone compounds and derivatives thereof.

### SUMMARY OF THE INVENTION

In one aspect, processes for dialkylating indolinone compounds are provided.

In another aspect, processes for dialkylating indolin-2-one compounds are provided.

In a further aspect, processes for dialkylating indolin-2-one compounds are provided which include reacting the indolinone with at least 2 equivalents of a first base to form the di-anion of the indolinone; and reacting the di-anion with an alkylating agent in the presence of a second base.

In yet another aspect, processes for dialkylating indolin-2-one compounds are provided which include dialkylating the indolin-2-one in the presence of at least 2 equivalents of a first base, a second base containing at least 1 equivalent of lithium diisopropylamide, and an alkylating agent.

In a further aspect, processes for preparing compounds of the following structure are provided, wherein R¹, R³, R⁴, R⁶, R⁷, R⁹, and R¹⁰ are as defined herein. 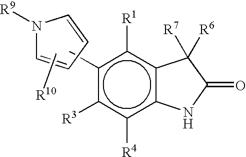
In still another aspect, a process for preparing 5-(4′-fluoro-2′-oxospiro[cyclopropane-1,3′-indoline]-5′-yl)-1-methyl-1H-pyrrole-2-carbonitrile is provided.

Other aspects and advantages of the invention will be readily apparent from the following detailed description of the invention.

### BRIEF DESCRIPTION OF THE DRAWINGS

FIG. 1 provides the X-ray diffraction (XRD) pattern for a sample of polymorph Form A 5-(4′-fluoro-2′-oxospiro[cyclopropane-1,3′-indoline]-5′-yl)-1-methyl-1H-pyrrole-2-carbonitrile.

FIG. 2 provides the differential scanning calorimetry (DSC) thermogram for a sample of polymorph Form A 5-(4′-fluoro-2′-oxospiro[cyclopropane-1,3′-indoline]-5′-yl)-1-methyl-1H-pyrrole-2-carbonitrile.

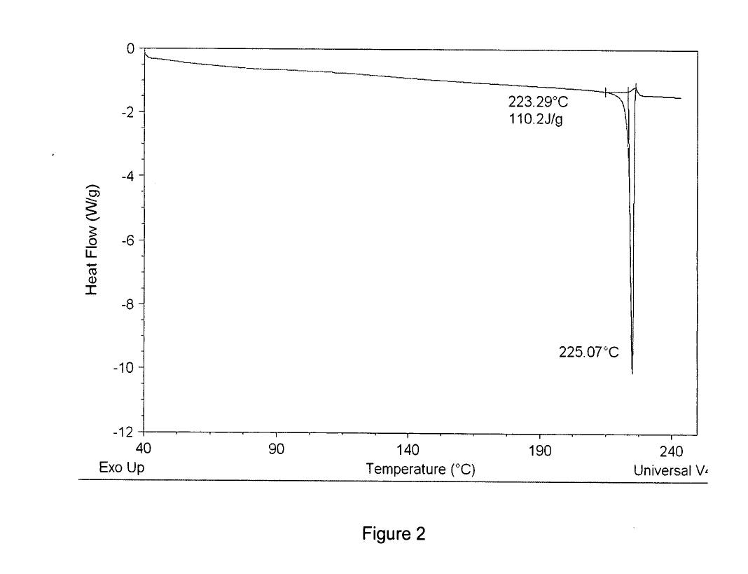

### DETAILED DESCRIPTION OF THE INVENTION

Processes for preparing progesterone receptor modulators are described. Specifically, processes for preparing indolinone compounds, particularly indolin-2-one compounds, are provided. These processes include protecting the amine group of an aniline compound, desirably with a t-butyloxy carbonyl (BOC) group; lithiating the methyl group attached to the carbon-atom adjacent to the BOC-protecting amine group; carboxylating the lithiated methyl group; deprotecting the amine group; cycloamidating to form the indolinone; dialkylating the indolinone at the 3-position in the presence of at least 2 equivalents of a first base, at least 1 equivalent of lithium diisopropylamide, and at least 2 equivalents of an alkylating agent; brominating the dialkyated indolinone; and coupling the brominated indolinone with a pyrrole compound. These processes are especially desirable for large scale preparations of the desired compounds. See, Scheme 2.

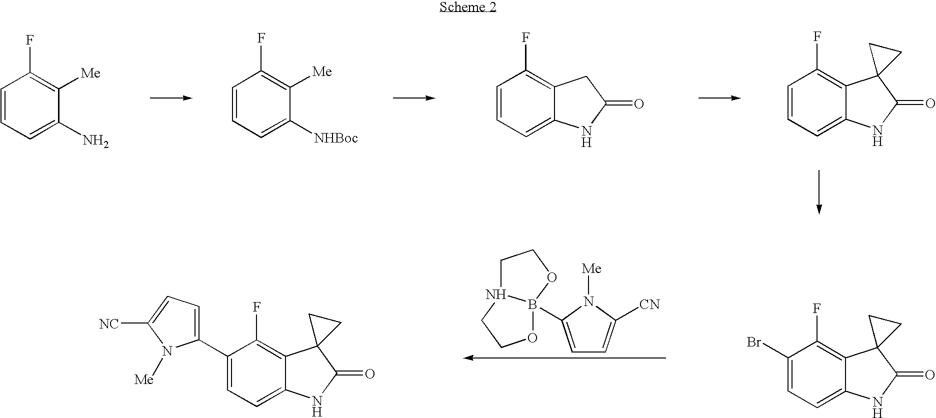

The inventors found that dialkylation of an indolinone at the 3-position to prepare a dialkylated indolinone and coupling of a dialkylated, brominated indolinone with a pyrrole compound to prepare a pyrrole substituted indolinone afforded high yields of the respective product, which steps are discussed individually below.

The term “dialkylated” or variations of this term as used herein describes the point of attachment of an alkyl group on an indolinone backbone. Desirably, “dialkylated” describes the point of attachment of an alkyl group at one carbon atom of an indolinone backbone. In one embodiment, the term “dialkylated” describes an indolinone that contains two alkyl groups attached to the same carbon atom. In another embodiment, “dialkylated” describes an indolinone that contains one alkyl group attached to a carbon atom of the indolinone through 2 separate carbon-atoms of the alkyl group.

### Dialkylation of Indolinones

The processes discussed herein provide efficient dialkylations of indolinone compounds. These processes thereby result in dialkylated indolinones and minimal, if any, side-products. By doing so, dialkylated indolinones of the following structure can be prepared: 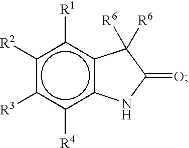
wherein, R¹, R², R³, and R⁴ are, independently, selected from among H, chlorine, fluorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂; wherein said C₂ to C₆ alkynyl and substituted C₂ to C₆ alkynyl groups of R¹ to R⁴ contain internal triple bonds; or R¹ and R²; R² and R³; R³ and R⁴; R¹, R², and R³; or R², R³, and R⁴ are fused to form (i) a 3 to 15 membered saturated or unsaturated carbon-containing ring; or (ii) a 3 to 15 membered heterocyclic ring containing in its backbone 1 to 3 heteroatoms selected from among O, S, and NR¹¹; R⁵ is selected from among C₁ to C₆ alkyl and C₁ to C₆ substituted alkyl; R¹¹ is absent, H, C₁ to C₆ alkyl, substituted C₁ to C₆ alkyl, aryl, or substituted aryl; R⁶ is selected from among C₁ to C₁₀ alkyl, substituted C₁ to C₁₀ alkyl, C₃ to C₁₄ cycloalkyl, or substituted C₃ to C₁₄ cycloalkyl; or the R⁶ groups are fused together to form a 3 to 8 membered saturated carbon-containing ring. In another embodiment, 5-(4′-fluoro-2′-oxospiro[cyclopropane-1,3′-indoline]-5′-yl)-1-methyl-1H-pyrrole-2-carbonitrile can be prepared according to the processes discussed herein.

The term “alkyl” is used herein to refer to both straight- and branched-chain saturated aliphatic hydrocarbon groups. In one embodiment, an alkyl group has 1 to 8 carbon atoms (i.e., C₁, C₂, C₃, C₄, C₅ C₆, C₇, or C₈). In another embodiment, an alkyl group has 1 to 6 carbon atoms (i.e., C₁, C₂, C₃, C₄, C₅ or C₆). In a further embodiment, an alkyl group has 1 to 4 carbon atoms (i.e., C₁, C₂, C₃, or C₄). Examples include methyl, ethyl, n-propyl, i-propyl, n-butyl, s-butyl, t-butyl, pentyl and hexyl, among others.

The term “cycloalkyl” is used herein to refer to cyclic, saturated aliphatic hydrocarbon groups. In one embodiment, a cycloalkyl group has 3 to 14 carbon atoms (i.e., C₃, C₄, C₅, C₆, C₇, C₈, C₉, C₁₀, C₁₁, C₁₂, C₁₃, or C₁₄). In a further embodiment, a cycloalkyl group has 3 to 8 carbon atoms (i.e., C₃, C₄, C₅, C₆, C₇, or C₈). In another embodiment, a cycloalkyl group has 3 to 6 carbon atoms (i.e., C₃, C₄, C₅ or C₆). Examples include cyclopropyl, cyclobutyl, cyclopentyl and cyclohexyl, among others.

The term “alkenyl” is used herein to refer to both straight- and branched-chain alkyl groups having one or more carbon-carbon double bonds. In one embodiment, an alkenyl group contains 3 to 8 carbon atoms (i.e., C₃, C₄, C₅, C₆, C₇, or C₈). In another embodiment, an alkenyl group has 1 or 2 carbon-carbon double bonds and 3 to 6 carbon atoms (i.e., C₃, C₄, C₅ or C₆). Examples include propenyl, among others.

The term “alkynyl” is used herein to refer to both straight- and branched-chain alkyl groups having one or more carbon-carbon triple bonds. In one embodiment, an alkynyl group has 3 to 8 carbon atoms (i.e., C₃, C₄, C₅, C₆, C₇, or C₈). In another embodiment, an alkynyl group contains 1 or 2 carbon-carbon triple bonds and 3 to 6 carbon atoms (i.e., C₃, C₄, C₅, or C₆). Examples include propynyl, among others.

The term “internal triple bond” as used herein refers to an alkynyl group whereby the triple bond is not connected to the last carbon atom of the alkynyl group, i.e., the carbon-atom that is not bound to the indolinone backbone. In summary, internal triple bond does not include an alkynyl moiety that contains a ˜C≡CH group.

The terms “substituted alkyl”, “substituted alkenyl”, “substituted alkynyl”, and “substituted cycloalkyl” refer to alkyl, alkenyl, alkynyl, and cycloalkyl groups, respectively, having one or more substituents e.g. 1 to 3 substituents which may be the same or different, selected from hydrogen, halogen, CN, OH, NO₂, amino, aryl, heterocyclyl, alkoxy, aryloxy, alkylcarbonyl, alkylcarboxy, and arylthio. One suitable group of substituents is hydrogen, halogen, CN, OH, NO₂, amino, phenyl, C₁-C₄ alkoxy, phenoxy, C₁-C₄ alkylcarbonyl, C₁-C₄ alkylcarboxy and phenylthio.

The term “arylthio” as used herein refers to the S(aryl) group, where the point of attachment is through the sulfur-atom and the aryl group can be substituted, e.g., by 1 to 4 substituents, the same or different, selected from among hydrogen, halogen, CN, OH, NO₂, amino, phenyl, C₁-C₄ alkoxy, phenoxy, C₁-C₄ alkylcarbonyl, C₁-C₄ alkylcarboxyl and phenylthio. The term “alkoxy” as used herein refers to the O(alkyl) group, where the point of attachment is through the oxygen-atom and the alkyl group can be substituted, e.g., by 1 to 4 substituents, the same or different, selected from among hydrogen, halogen, CN, OH, NO₂, amino, phenyl, C₁-C₄ alkoxy, phenoxy, C₁-C₄ alkylcarbonyl, C₁-C₄ alkylcarboxyl and phenylthio. The term “aryloxy” as used herein refers to the O(aryl) group, where the point of attachment is through the oxygen-atom and the aryl group can be substituted, e.g., by 1 to 4 substituents, the same or different, selected from among hydrogen, halogen, CN, OH, NO₂, amino, phenyl, C₁-C₄ alkoxy, phenoxy, C₁-C₄ alkylcarbonyl, C₁-C₄ alkylcarboxyl and phenylthio.

The term “alkylcarbonyl” as used herein refers to the C(O)(alkyl) group, where the point of attachment is through the carbon-atom of the carbonyl moiety and the alkyl group can be substituted, e.g., by 1 to 4 substituents, the same or different, selected from among hydrogen, halogen, CN, OH, NO₂, amino, phenyl, C₁-C₄ alkoxy, phenoxy, C₁-C₄ alkylcarbonyl, C₁-C₄ alkylcarboxyl and phenylthio.

The term “alkylcarboxy” as used herein refers to the C(O)O(alkyl) group, where the point of attachment is through the carbon-atom of the carboxy moiety and the alkyl group can be substituted, e.g., by 1 to 4 substituents, the same or different, selected from among hydrogen, halogen, CN, OH, NO₂, amino, phenyl, C₁-C₄ alkoxy, phenoxy, C₁-C₄ alkylcarbonyl, C₁-C₄ alkylcarboxyl and phenylthio.

The term “alkylamino” as used herein refers to both secondary and tertiary amines where the point of attachment is through the nitrogen-atom and the alkyl groups can be substituted, e.g., by 1 to 4 substituents, the same or different, selected from hydrogen, halogen, CN, OH, NO₂, amino, phenyl, C₁-C₄ alkoxy, phenoxy, C₁-C₄ alkylcarbonyl, C₁-C₄ alkylcarboxyl and phenylthio. The alkyl groups can be the same or different.

The term “halogen” as used herein refers to Cl, Br, F, or I.

The term “aryl” as used herein refers to an aromatic, carbocyclic system, e.g., of 6 to 14 carbon atoms, which can include a single ring or multiple aromatic rings fused or linked together where at least one part of the fused or linked rings forms the conjugated aromatic system. The aryl groups include, but are not limited to, phenyl, naphthyl, biphenyl, anthryl, tetrahydronaphthyl, phenanthryl, indene, benzonaphthyl, and fluorenyl.

The term “substituted aryl” refers to an aryl group which is substituted with one or more substituents selected from halogen, CN, OH, NO₂, amino, alkyl, cycloalkyl, alkenyl, alkynyl, C₁ to C₃ perfluoroalkyl, C₁ to C₃ perfluoroalkoxy, aryloxy, alkoxy including —O— (C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl. Desirably, a substituted aryl group is substituted with 1 to 4 substituents which may be the same or different.

The term “heterocycle” or “heterocyclic” as used herein can be used interchangeably to refer to a stable, saturated or partially unsaturated 3- to 9-membered monocyclic or multicyclic heterocyclic ring. The heterocyclic ring has in its backbone carbon atoms and one or more heteroatoms including nitrogen, oxygen, and sulfur atoms. In one embodiment, the heterocyclic ring has 1 tot 4 heteroatoms in the backbone of the ring. When the heterocyclic ring contains nitrogen or sulfur atoms in the backbone of the ring, the nitrogen or sulfur atoms can be oxidized. The term “heterocycle” or “heterocyclic” also refers to multicyclic rings in which a heterocyclic ring is fused to an aryl ring of 6 to 14 carbon atoms. The heterocyclic ring can be attached to the aryl ring through a heteroatom or carbon atom provided the resultant heterocyclic ring structure is chemically stable. In one embodiment, the heterocyclic ring includes multicyclic systems having 1 to 5 rings. Suitable heterocyclic rings include those having 6 to 12, preferably 6 to 10 ring members containing 1 to 3 heteroatoms selected from N, O and S. Suitable heteroaryl rings include those having 5 to 12 preferably 5 to 10 ring members containing 1 to 3 heteroatoms selected from N, O and S.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 14-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to 4 heteroatoms in the backbone of the ring which may suitably be selected from O, S and N. When the heteroaryl ring contains nitrogen or sulfur atoms in the backbone of the ring, the nitrogen or sulfur atoms can be oxidized. The term “heteroaryl” also refers to multicyclic rings in which a heteroaryl ring is fused to an aryl ring. The heteroaryl ring can be attached to the aryl ring through a heteroatom or carbon atom provided the resultant heterocyclic ring structure is chemically stable. In one embodiment, the heteroaryl ring includes multicyclic systems having 1 to 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents, the same or different selected from halogen, CN, OH, NO₂, amino, alkyl, cycloalkyl, alkenyl, alkynyl, C₁ to C₃ perfluoroalkyl, C₁ to C₃ perfluoroalkoxy, aryloxy, alkoxy including —O—(C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl. A substituted heterocycle or heteroaryl group may have 1, 2, 3, or 4 substituents.

A variety of indolinones can be dialkylated using the processes described herein. In one embodiment, the indolinone is an indolin-2-one, which is dialkylated at the 3-position. In another embodiment, the indolinone is of the following structure, which is dialkylated at the 3-position: 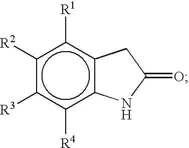
wherein, R¹, R², R³, and R⁴ are as defined above. In a further embodiment, the indolinone is 4-fluoroindolin-2-one.

The inventors found that the most efficient dialkylation of an indolin-2-one at the 3-position is performed when the corresponding dianion of the indolinone is prepared and thereby is reacted with an alkylating agent. In one embodiment, the indolin-2-one dianion is formed prior to addition of the alkylating agent. In another embodiment, the indolin-2-one dianion is formed in the presence of the alkylating agent. Dialkylation of the 3-position of the indolinone is performed in the absence of N-alkylation.

The dialkylation includes preparing a dianion of an indolin-2-one using a first base, reacting the dianion with an alkylating agent in the presence of at least one equivalent of a second base. Desirably the second base is lithium diisopropylamide. See, Scheme 3, wherein R¹-R⁴, and R⁶ are defined herein.

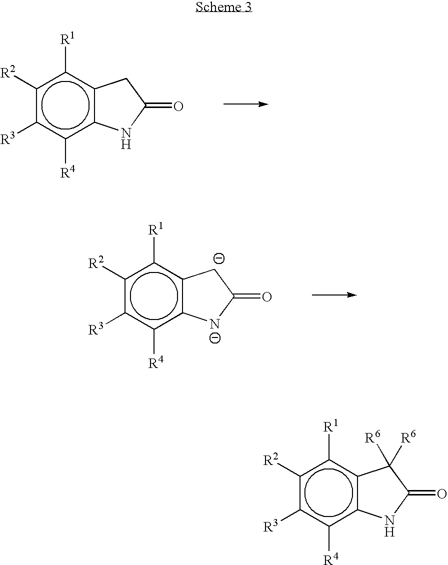

As discussed above, the first base is utilized to generate the indolin-2-one dianion. The base must be sufficiently strong to generate the dianion of the indolin-2-one. The first base may therefore be an alkyl lithium, an alkali metal hydride, a Grignard reagent, an alkali metal alkyl amide, an alkali metal disilazide, or mixtures thereof.

In one embodiment, the first base is a Grignard reagent such as R¹²MgX¹, wherein X¹ is chlorine, bromine, or iodine and R¹² is C₁ to C₆ alkyl, substituted C₁ to C₆ alkyl, aryl, or substituted aryl.

In another embodiment, the first base is an alkali metal hydride such as sodium hydride, potassium hydride, or lithium hydride.

In a further embodiment, the first base is an alkali metal alkyl amide such as (R¹⁴)(R¹⁵)N-M, wherein R¹⁴ and R¹⁵ are, independently, H, C₁ to C₆ alkyl or substituted C₁ to C₆ alkyl or R¹⁴ and R¹⁵ are fused to form a carbon-based 3 to 8 membered saturated ring and M is lithium, sodium, or potassium.

In still a further embodiment, the first base is an alkali metal disilazide such as ((R¹³)₃Si)₂N-M, wherein, R¹³ is C₁ to C₆ alkyl or substituted C₁ to C₆ alkyl and M is lithium, sodium, or potassium.

In yet another embodiment, the first base is an alkyl lithium such as a C₁ to C₁₀ alkyl lithium. Desirably, the alkyl lithium is butyl lithium, and more desirably n-butyl lithium. At least about 2 equivalents of alkyl lithium are required. In one embodiment, about 2 equivalents of alkyl lithium are utilized. In another embodiment, about 3 equivalents of alkyl lithium are utilized. The first base, i.e., alkyl lithium, is added prior to the dialkylation.

In still another embodiment, the first base is an alkali metal alkyl amide including lithium diisopropylamide (LDA). The LDA can be added to the reaction mixture or generated in situ. If the LDA is generated in situ, it is desirable that it is generated prior to the dialkylation. Typically, the LDA is generated using an alkyl lithium, such as those described above, and diisopropylamine. Desirably, the LDA is generated using 2 equivalents of an alkyl lithium and 2 equivalents of diisopropylamine. More desirably, the LDA is generated using 2 equivalents of butyl lithium and 2 equivalents of diisopropylamine. The LDA is added to the reaction mixture before the alkylating agent.

Typically, the indolin-2-one dianion is generated at temperatures less than about room temperature. In one embodiment, the indolin-2-one dianion is generated at a temperature of about −90 to about 25° C. In another embodiment, the indolin-2-one dianion is generated at a temperature of about −40 to about 0° C.

It is also necessary for at least one or more equivalents of a second base, i.e., additional to what is required to form the dianion, to be present to effect high yields of the dialkylated indolin-2-one. Desirably, the second base reacts very slowly with the alkylating agent or does not react with the alkylating agent. In one embodiment, the second base is added to the reaction mixture concurrently with the alkylating agent. In a further embodiment, the second base is added to the reaction mixture prior to the alkylating agent.

Typically, the second base is a hindered amide base. In one embodiment, the second base is a metal alkyl amide. In another embodiment, the second base is (R¹⁴)(R¹⁵)N-M, wherein R¹⁴, R¹⁵, and M are defined above. In a further embodiment, the second base is LDA. Desirably, the first and second bases are LDA. In one embodiment, at least 1 equivalent of the second base is present. In another embodiment, at least 1.1 equivalents of the second base are present. In a further embodiment, about 2 equivalents of the second base are present.

In one embodiment, the second base is the same as the first base. In another embodiment, the second base differs from the first base. The second base may be generated in situ or added to the reaction. In one embodiment, the second base is generated in situ during preparation of the dianion. In another embodiment, the second base is added as a separate component of the reaction before the dianion is prepared. In a further embodiment, the second base is added to the solution as a separate component while the dianion is being prepared. In yet another embodiment, the second base is added to the solution as a separate component after the dianion has been prepared. In still a further embodiment, the second base is added to the process simultaneously with the first base. In another embodiment, the second base is added to the process separately from the first base.

In order to perform the dialkylation, an alkylating agent must be utilized. A variety of alkylating agents are useful in the processes described herein. See, Larock, “Comprehensive Organic Transformations”, VCH Publishers, Inc., New York, N.Y., 1989, which is hereby incorporated by reference. Desirably, the alkylation is performed at about −40° C. to about 50° C. More desirably, the alkylation is performed at about 0° C. to about 50° C. Even more desirably, the alkylation is performed at about 15° C. to about 30° C.

In one embodiment, the alkylating agent is a mono-alkylating agent. The term “mono-alkylating agent” as used herein refers to an alkylating agent that contains an alkyl group that binds to the indolinone through one carbon-atom of the alkyl group. In one embodiment, the mono-alkylating agent is R⁶X², wherein R⁶ is C₁ to C₁₀ alkyl, substituted C₁ to C₁₀ alkyl, C₃ to C₁₄ cycloalkyl, or substituted C₃ to C₁₄ cycloalkyl; X² is halogen or OSO₂R¹⁶; and R¹⁶ is C₁ to C₁₀ alkyl, substituted C₁ to C₁₀ alkyl, aryl, or substituted aryl. Desirably, the mono-alkylating agent is methyl iodide, methyl bromide, or ethyl bromide. Most desirably, the mono-alkylating agent is methyl iodide. At least 2 equivalents of the mono-alkylating agent are utilized. In one embodiment, at least 2.1 equivalents of the mono-alkylating agent are utilized. In a further embodiment, about 2.1 to about 10 equivalents of the mono-alkylating agent are utilized. In another embodiment, about 2.1 equivalents of the mono-alkylating agent are utilized.

In another embodiment, the alkylating agent is a “di-alkylating agent”. The term “di-alkylating agent” as used herein refers to an alkylating agent that contains an alkyl group which binds to the indolinone through two carbon atoms of the alkyl group. In one embodiment, the di-alkylating agent is X³—(CH₂)ₙ—X⁴, wherein X³ and X⁴ are, independently, Cl, Br, or OSO₂R¹⁶; R¹⁶ is C₁ to C₁₀ alkyl, substituted C₁ to C₁₀ alkyl, aryl, or substituted aryl; and n is 2 to 7. In another embodiment, the di-alkylating agent is 1,2-dibromoethane, 1,2-dichloroethane, 1-bromo-2-chloroethane, 1,3-dibromopropane, 1,4-dibromobutane, or 1,5-dibromopentane. Desirably, at least about 1 equivalent of a di-alkylating agent are utilized. More desirably, at least about 1.1 equivalents of a di-alkylating agent are utilized. Even more desirably, about 1 to about 10 equivalents of a di-alkylating agent are utilized. Most desirably, about 1 to about 3 equivalents of a di-alkylating agent are utilized.

By performing the dialkylation as described herein, the dialkylated indolinone is prepared at a greater than a 90% yield, greater than a 91% yield, greater than a 92% yield, greater than a 93% yield, greater than a 94% yield, greater than a 95% yield, greater than a 96% yield, greater than a 97% yield, greater than a 98% yield, or greater than a 99% yield. Desirably, the dialkylated indolinone is prepared in a 100% yield.

In one embodiment, a process for dialkylating an indolin-2-one is provided and includes reacting the indolin-2-one with at least 2 equivalents of a first base to form the di-anion of the indolinone; and reacting the di-anion with an alkylating agent in the presence of a second base.

In a further embodiment, a process for dialkylating an indolin-2-one is provided and includes performing the dialkylation in the presence of at least 2 equivalents of a first base, a second base containing at least 1 equivalent of lithium diisopropylamide, and an alkylating agent.

In another embodiment, a process for dialkylating an indolinone compound of the following structure at the 3-position is provided: 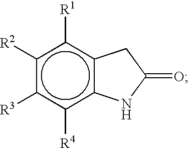
wherein, R¹ to R⁴ are defined above. The process includes reacting the dianion of an indolin-2-one with an alkylating agent in the presence of a base containing at least 1 equivalent of lithium diisopropylamide. Desirably, the dianion is prepared by reacting the indolin-2-one with 2 equivalents of a first base.

In a further embodiment, a process for dialkylating an indolinone compound of the following structure at the 3-position is provided: 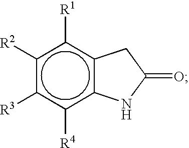
wherein, R¹ to R⁴ are defined above. The process includes dialkylating the indolinone in the presence of at least 2 equivalents of a first base, a second base containing at least 1 equivalent of lithium diisopropylamide, and an alkylating agent.

In another embodiment, a process is provided for dialkylating an indolinone compound of the following structure at the 3-position: 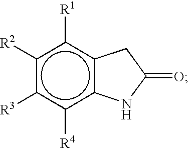
wherein, R¹ to R⁴ are defined above. The process includes dialkylating the indolinone in the presence of at least 3 equivalents of lithium diisopropylamide and an alkylating agent.

In another embodiment, a process is provided for dialkylating an indolinone compound of the following structure at the 3-position: 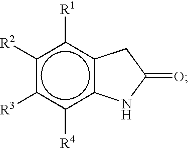
wherein, R¹ to R⁴ are defined above. The process includes reacting the indolinone with 2 equivalents of butyl lithium to form the indolinone dianion and reacting the dianion with an alkylating agent in the presence of lithium diisopropyl amide.

In a further embodiment, a process is provided for dialkylating an indolinone compound of the following structure at the 3-position: 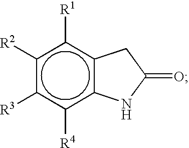
wherein, R¹ to R⁴ are defined above. The process includes reacting the indolinone with about 2 equivalents of lithium diisopropylamide to form the indolinone dianion and reacting the dianion with an alkylating agent in the presence of at least about 1.1 equivalents of lithium diisopropylamide.

In yet another embodiment, a process for preparing 4′-Fluorospiro[cyclopropane-1,3′-indolin]-2′-one is provided and includes (i) reacting 4-fluoroindolin-2-one and 2 equivalents of butyl lithium and (ii) reacting 1,2-dibromoethane and lithium diisopropylamide with the product of step (i).

In a further embodiment, a process for preparing 4′-Fluorospiro[cyclopropane-1,3′-indolin]-2′-one is provided and includes (i) reacting 4-fluoroindolin-2-one and lithium diisopropylamide; and (ii) reacting 1,2-dibromoethane and lithium diisopropylamide with the product of step (i).

### Processes for Coupling Indolinones and Pyrrole Reagents

Also described herein are processes for preparing pyrrole coupled indolinones. In one embodiment, processes for preparing 5-pyrrole-indolin-2-ones are provided. In another embodiment, compounds of the following structure are prepared using the processes described herein. 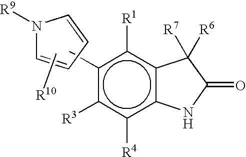
wherein, R¹, R³, and R⁴ are, independently, selected from among H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂; or R³ and R⁴ are fused to form (i) a 3 to 15 membered saturated or unsaturated carbon-containing ring; or (ii) a 3 to 15 membered heterocyclic ring containing in its backbone from 1 to 3 heteroatoms selected from the group consisting of O, S, and NR¹¹; R⁵ is selected from among C₁ to C₆ alkyl and C₁ to C₆ substituted alkyl; R⁶ and R⁷ are, independently, selected from among C₁ to C₆ alkyl, substituted C₁ to C₆ alkyl, C₃ to C₁₄ cycloalkyl, substituted C₃ to C₁₄ cycloalkyl, aryl substituted aryl, heteroaryl, substituted heteroaryl, C₃ to C₆ alkenyl, substituted C₃ to C₆ alkenyl, C₃ to C₆ alkynyl, substituted C₃ to C₆ alkynyl, SR⁵, OR⁵, and N(R⁵)₂; or R⁶ and R⁷ are fused to form a 3 to 8 membered saturated carbon-containing ring; R⁹ is selected from among C₁ to C₆ alkyl, substituted C₁ to C₆ alkyl, aryl, substituted aryl, heteroaryl, substituted heteroaryl, and COR^{A}; R^{A} is selected from among H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl; R¹⁰ is selected from among H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A}; and R¹¹ is absent, H, C₁ to C₆ alkyl, substituted C₁ to C₆ alkyl, aryl, or substituted aryl. Desirably, 5-(4′-fluoro-2′-oxospiro[cyclopropane-1,3′-indoline]-5′-yl)-1-methyl-1H-pyrrole-2-carbonitrile is prepared according to the process.

The inventors found that an indolinone and pyrrole compound could be coupled using the diethanolamine complex of a pyrrole boronic acid. See, US Patent Application Publication No. US-2005-0272702-A1, which is hereby incorporated by reference, and describes the preparation of diethanolamine complexes of pyrrole boronic acids. Specifically, since the diethanolamine complex of the pyrrole boronic acid is more stable than the pyrrole boronic acid alone, higher yields of the pyrrole coupled indolin-2-one could be prepared. The inventors also determined that as little as 1.2 equivalents of the diethanolamine complex of the pyrrole boronic acid were necessary, as opposed to the greater than 2 equivalents of pyrrole boronic acid. By doing so, chromatography was not required to isolate the coupled pyrrole indolin-2-one. However, greater than 1.2 equivalents of the diethanolamine complex of the pyrrole boronic acid may be utilized.

The processes of preparing the pyrrole indolinone compounds thereby include reacting a compound of the following structure, wherein R¹, R³, R⁴, R⁶, and R⁷ are defined above: 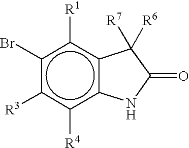
with a pyrrole compound of the structure: 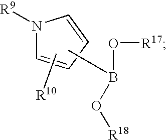
wherein, R⁹ and R¹⁰ are defined above, R¹⁷ and R¹⁸ are, independently, H, C₁ to C₆ alkyl, or substituted C₁ to C₆ alkyl; or R¹⁷ and R¹⁸ are fused to form (i) a saturated carbon-atom based 5 to 10 membered ring; or (ii) a saturated carbon-atom based 5 to 8 membered ring containing one or more heteroatoms selected from among O, S, and NR¹⁹; R¹⁹ is H, C₁ to C₆ alkyl, or substituted C₁ to C₆ alkyl; in the presence of a palladium catalyst containing a phosphine ligand. In one embodiment, R¹⁷ and R¹⁸ are fused to form —(CR²⁰₂)—(CH₂)ₙ—(CR²⁰₂)—, where n is 0 to 6 and R²⁰ is, independently, H or C₁ to C₆ alkyl. In one embodiment, R²⁰ is methyl. In another embodiment, R¹⁷ and R¹⁸ are fused to form —(CH₂)ₘ—(NR¹⁹)—(CH₂)_{q}—; m is 1 to 6; and q is 1 to 6. In a further embodiment, the pyrrole compound is a diethanolamine complex of the following structure, wherein R⁹ and R¹⁰ are defined above:

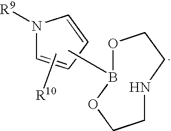

A variety of palladium catalysts that contain phosphine ligands and are useful to effect coupling of the indolinone and pyrrole compound are described in Negishi et al., “Handbook of Organopalladium Chemistry for Organic Synthesis”, Wiley: New York, N.Y. (2002) and Diederich et al., “Metal-Catalyzed Cross-Coupling Reactions”, Wiley: New York, N.Y. (1998), which are both hereby incorporated by reference. One of skill in the art would readily be able to select a suitable palladium catalyst. The palladium catalyst utilized in the process may be commercially available and includes Pd(PPh₃)₄, Pd(PPh₃)₂Cl₂, or Pd(dppf)Cl₂, without limitation. In one embodiment, the palladium catalyst is added to the process as a separate reagent. In another embodiment, the palladium catalyst in generated in situ using a palladium reagent and a phosphine reagent. Desirably, the palladium catalyst is present at an amount of about 0.1 to about 10 mol %. More desirably, the palladium catalyst is present at an amount of about 0.5 to about 3 mol %. Most desirably, about 2 mol % of the palladium catalyst is utilized.

Alternatively, the palladium catalyst can be prepared by reacting a palladium reagent with a phosphine reagent or a salt thereof. The term “palladium reagent” describes a chemical compound that contains palladium and reacts with a phosphine reagent to form the palladium catalyst described herein. Several palladium reagents are useful to generate the palladium catalyst utilized as described herein and include Pd₂(dba)₃ and Pd(OAc)₂, among others. See, e.g., the palladium reagents provided in the catalog by Strem Chemicals, Inc. and in Negishi cited above, which are hereby incorporated by reference herein.

The term “phosphine reagent” describes a chemical compound that contains phosphorus and can react with the palladium catalyst described above. Several phosphine reagents are useful in this process and may be selected by one of skill in the art. See, e.g., the phosphine reagents provided in the catalog by Strem Chemicals, Inc. and in Negishi cited above. Desirably, phosphine reagents that can be utilized include tri-t-butylphosphine or a salt thereof. In one embodiment, the phosphine salt is tri-t-butylphosphine hydrotetrafluoroborate. In one embodiment, the palladium reagent reacts with tri-t-butylphosphine hydrotetrafluoroborate to form Pd[P(^{t}Bu)₃] as the palladium catalyst. Desirably, the ratio of palladium reagent to phosphine reagent, or salt thereof, is about 1:1 to about 1:4. More desirably, the ratio of palladium reagent to phosphine reagent, or salt thereof, is 1:1 to 1:1.5.

The process of coupling the indolin-2-one and pyrrole reagent may be performed in the presence of a mild base, such as those provided in Negishi and Diederich cited above and hereby incorporated by reference. The term “mild base” as used herein refers to a base that is capable of suppressing or eliminating the decomposition of the pyrrole compound boronic acid. In one embodiment, the mild base is selected from among an alkali bicarbonate, an alkali phosphate, an alkali hydrophosphate, an alkali fluoride, and an alkali acetate, without limitation. In another embodiment, the mild base is an alkali bicarbonate. In a further embodiment, the mild base is sodium bicarbonate. In another embodiment, the mild base is potassium phosphate. Desirably, about 1 to 3 equivalents of the mild base are utilized. More desirably, about 3 equivalents of the mild base are utilized.

A number of solvents can be utilized in the coupling process and may be selected by one of skill in the art. Desirably, the solvent is any inert solvent that is capable of partially or completely dissolving the indolinone and pyrrole compound. Desirably, the solvent is dimethoxyethane (DME), tetrahydrofuran (THF), dimethylacetamide (DMA), dimethylformamide (DMF), N-methylpyrrolidone (NMP), or combinations thereof, among others.

The coupling processes are typically performed at temperatures greater than about 0° C. Desirably, the coupling processes are performed at a temperature of about 0° C. to about the reflux temperature of the solvent. One of skill in the art would readily be able to determine the temperature for the coupling process given the selected solvent, reagents, environmental conditions, among other factors.

By performing the coupling as described above, the pyrrole coupled indolinone can be prepared at a yield of greater than 60%, greater than 75%, and greater than 90%.

In one embodiment, a process is provided for preparing a compound of the structure: 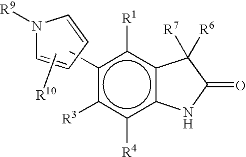
wherein, R¹, R³, and R⁴ are, independently, selected from among H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂; or R³ and R⁴ are fused to form (i) a 3 to 15 membered saturated or unsaturated carbon-containing ring; or (ii) a 3 to 15 membered heterocyclic ring containing in its backbone from 1 to 3 heteroatoms selected from the group consisting of O, S, and NR¹¹; R⁵is selected from among C₁ to C₆ alkyl and C₁ to C₆ substituted alkyl; R⁶ and R⁷ are, independently, selected from among C₁ to C₆ alkyl, substituted C₁ to C₆ alkyl, C₃ to C₁₄ cycloalkyl, substituted C₃ to C₁₄ cycloalkyl, aryl substituted aryl, heteroaryl, substituted heteroaryl, C₃ to C₆ alkenyl, substituted C₃ to C₆ alkenyl, C₃ to C₆ alkynyl, substituted C₃ to C₆ alkynyl, SR⁵, OR⁵, and N(R⁵)₂; or R⁶ and R⁷ are fused to form a 3 to 8 membered saturated carbon-containing ring; R⁹is selected from among C₁ to C₆ alkyl, substituted C₁ to C₆ alkyl, aryl, substituted aryl, heteroaryl, substituted heteroaryl, and COR^{A}; R^{A} is selected from among H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl; R¹⁰ is selected from among H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A}; and R¹¹ is absent, H, C₁ to C₆ alkyl, substituted C₁ to C₆ alkyl, aryl, or substituted aryl. The process includes reacting a compound of the structure: 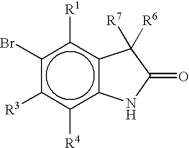
with a pyrrole compound of the structure: 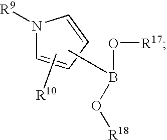
wherein, R¹⁷ and R¹⁸ are, independently, H, C₁ to C₆ alkyl, or substituted C₁ to C₆ alkyl; or R¹⁷ and R¹⁸ are fused to form (i) a saturated carbon-atom based 5 to 10 membered ring; or (ii) a saturated carbon-atom based 5 to 8 membered ring containing one or more heteroatoms selected from among O, S, and NR¹⁹; R¹⁹ is H, C₁ to C₆ alkyl, or substituted C₁ to C₆ alkyl; in the presence of a palladium catalyst containing a phosphine ligand.

In another embodiment, a process is provided which comprises reacting a compound of the structure: 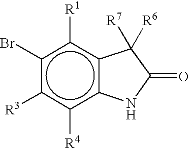
as defined above, with the diethanolamine complex of 5-cyano-1-methyl-1H-pyrrol-2-ylboronic acid in the presence of a palladium catalyst containing a phosphine ligand.

In a further embodiment, a process is provided for preparing 5-(4′-fluoro-2′-oxospiro[cyclopropane-1,3′-indoline]-5′-yl)-1-methyl-1H-pyrrole-2-carbonitrile. The process includes reacting 5′-bromo-4′-fluorospiro[cyclopropane-1,3′-indolin]-2′-one and the diethanolamine complex of 5-cyano-1-methyl-1H-pyrrol-2-ylboronic acid in the presence of a palladium catalyst containing a phosphine ligand.

In still another embodiment, a process is provided for preparing 5-(4′-fluoro-2′-oxospiro[cyclopropane-1,3′-indoline]-5′-yl)-1-methyl-1H-pyrrole-2-carbonitrile. The process includes reacting 5′-bromo-4′-fluorospiro[cyclopropane-1,3′-indolin]-2′-one and the diethanolamine complex of 5-cyano-1-methyl-1H-pyrrol-2-ylboronic acid in the presence of P(t-Bu)₃.HBF₄ and a palladium catalyst containing a phosphine ligand.

In yet a further embodiment, a process is provided for preparing 5-(4′-fluoro-2′-oxospiro[cyclopropane-1,3′-indoline]-5′-yl)-1-methyl-1H-pyrrole-2-carbonitrile. The process includes reacting 5′-bromo-4′-fluorospiro[cyclopropane-1,3′-indolin]-2′-one and the diethanolamine complex of 5-cyano-1-methyl-1H-pyrrol-2-ylboronic acid in the presence of about 1.1 equivalents of P(t-Bu)₃.HBF₄, NaHCO₃, about 2 mol % of Pd₂(dba)₃, and solvent containing DME and water.

### Processes for Preparing Pyrrole Coupled Indolinones From Anilines

The processes described herein also provide for preparing compounds of the following structure. As opposed to previous processes, which include at least six steps, the pyrrole coupled indolinone compounds herein are advantageously prepared using the described processes via only 5 steps. Further, the processes described herein only require the isolation of 4 intermediate compounds. 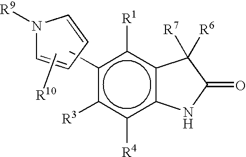
wherein, R¹, R³, and R⁴ are, independently, selected from among H, chlorine, C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, CF₃, SR⁵, OR⁵, N(R⁵)₂, and SO₂N(R⁵)₂; or R¹ and R²; R² and R³; R³ and R⁴; R¹, R², and R³; or R², R³, and R⁴ are fused to form: (a) a 3 to 15 membered saturated or unsaturated carbon-containing ring; or (b) a 3 to 15 membered heterocyclic ring containing in its backbone from 1 to 3 heteroatoms selected from the group consisting of O, S, and NR¹¹; R⁵ is selected from among C₁ to C₆ alkyl and C₁ to C₆ substituted alkyl; R⁶ and R⁷ are, independently, the same and are selected from among C₁ to C₆ alkyl, substituted C₁ to C₆ alkyl, C₃ to C₁₄ cycloalkyl, substituted and C₃ to C₁₄ cycloalkyl; or R⁶ and R⁷ are fused to form a 3 to 8 membered saturated carbon-containing ring; R⁹ is selected from among C₁ to C₆ alkyl, substituted C₁ to C₆ alkyl, aryl, substituted aryl, heteroaryl, substituted heteroaryl, and COR^{A}; R^{A} is selected from among H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl; R¹⁰ is selected from among H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A}; and R¹¹ is absent, H, C₁ to C₆ alkyl, substituted C₁ to C₆ alkyl, aryl, or substituted aryl.

The processes thereby first include protecting an aniline of the structure, where R¹, R³, and R⁴ are defined above.

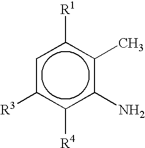

Protection of the aniline, i.e., protection of the amine group, is performed using a protecting group. The term “protecting group” as used herein includes any group that can serve as a lithiation-directing group. A variety of protecting groups can be selected by one of skill in the art and include those described in Gschwend et al., Org. React., 26:1 (1969), which is hereby incorporated by reference. Examples of suitable protecting groups include, without limitation, a carbamate such as —NHCOR²¹ (R²¹ is C₁ to C₁₀ alkyl or substituted C₁ to C₁₀ alkyl) or an amide such as —NHR²² (R²² is C₁ to C₁₀ alkyl or substituted C₁ to C₁₀ alkyl, preferably, tert-alkyl), among others. Desirably, the protecting group is a carbamate. More desirably, the protecting group is a t-butyloxy carbonyl (BOC) group. A variety of solvents can be utilized during the protection, provided that the solvent is aprotic and is capable of completely or partially dissolving the starting aniline. One of skill in the art could readily select a suitable solvent given the reagents utilized, among others. Examples of useful solvents include, without limitation, THF, toluene, methyl tert-butyl ether (MTBE), dichloroethane, acetonitrile, ethyl acetate, or combinations thereof, among others. Typically, the protection is performed at elevated temperatures. More typically, the protection is performed at about 50° C. to about the reflux temperature of the selected solvent. In one example, the aniline can be Boc-protected by heating the aniline with a slight excess of Boc anhydride in THF at reflux for about 10 to 20 hrs.

The protected aniline is then lithiated using a lithiating reagent. Desirably, the protected aniline is lithiated at the carbon-atom adjacent to the protected amine group. More desirably, the methyl group of the aniline is lithiated. Typically, the lithiating reagent is an alkyl lithium. Desirably, the alkyl lithium is butyl lithium. The alkyl lithium can contain an additive that enhances the reactivity of the lithiating reagent. Examples of additives that can utilized include, without limitation, tetramethylethylenediamine (TMEDA), 1,4-diazabicyclo[2.2.2]octane (DABCO), or hexamethyl phosphoramide (HMPA). The lithiation is performed in an inert solvent that does not react with the lithiated product. Desirably, the solvent is an ether. More desirably, the solvent is THF, ethyl ether, dimethoxyethane, or combinations thereof. The solvent may optionally be combined with one or more of a hydrocarbon solvent including, without limitation, heptane, hexane, or a combination thereof. Desirably, the lithiation is performed at reduced temperatures. In one embodiment, the lithiation is performed at temperatures less than about 0° C. In another embodiment, the lithiation is performed a temperature of about −90° C. to about 0° C. In a further embodiment, the lithiation is performed at a temperature of about −40° C. to about −10° C. In one example, the Boc-protected aniline is lithiated using a sec-BuLi solution in THF at a temperature of about −40° C. or below, the mixture is briefly warmed to about −18° C., cooled to reduced temperatures, and transferred onto a slurry of dry ice and THF. Typically, the lithiated product is utilized without further purification.

The lithiated aniline is then carboxylated using techniques known to those of skill in the art. Typically, the carboxylation is performed using carbon dioxide or Y¹C(O)X⁵; wherein, X⁵ and Y¹ are independently chlorine, bromine, C₁ to C₆ alkoxy, or substituted C₁ to C₆ alkoxy. In one embodiment, the carboxylation is performed using gaseous carbon dioxide. In another embodiment, the carboxylation is performed using solid carbon dioxide. The carboxylation is performed in an inert solvent that does not react with the starting materials or product. Desirably, the solvent is an ether. More desirably, the solvent is THF, ethyl ether, dimethoxyethane, or combinations thereof. The solvent may optionally be combined with one or more of a hydrocarbon including, without limitation heptane, hexane, or a combination thereof. Desirably, the carboxylation is performed at reduced temperatures. In one embodiment, the carboxylation is performed at temperatures less than about 0° C. In another embodiment, the carboxylation is performed a temperature of about −90° C. to about 0° C. In a further embodiment, the carboxylation is performed at a temperature of about −40° C. to about −10° C.

The protected, carboxylated aniline is then deprotected and cyclized using reagents known to those of skill in the art. Advantageously, these steps can be performed using the same reagent. Typically, these steps are performed using a strong inorganic or organic acid such as, without limitation, HCl, H₂SO₄, R²³—SO₃H (R²³ is C₁ to C₁₀ alkyl or substituted C₁ to C₁₀ alkyl), trifluoroacetic acid (TFA), triflic acid (TfOH), or combinations thereof. See, Green, T. W., Wuts, P.G.M. Protecting groups in organic chemistry, 3rd Ed., 1999, p. 494-615, which is hereby incorporated by reference. The deprotection/cyclization is performed in any inert solvent. Desirably, the solvent includes, without limitation, water, dioxane, diethyl ether, or mixtures thereof. Typically, the deprotection and cyclization is performed at elevated temperatures. In one embodiment, the deprotection and cyclization is performed at a temperature of greater than about 30° C. In another embodiment, the deprotection and cyclization is performed at a temperature of about 30° C. to the reflux temperature of the solvent. In a further embodiment, the deprotection and cyclization is performed at a temperature of about 50° C. to about 70° C. By doing so, a compound of the following structure can be prepared, wherein R¹, R³, and R⁴ are defined above. In one embodiment, the carboxylated product is treated with aqueous HCl solution to effect deprotection and cyclization in one step to afford the indolinone. Typically, the indolinone is prepared at an overall yield, including the protecting, lithiating, deprotecting, and cyclizing steps, of about 92 to about 96%.

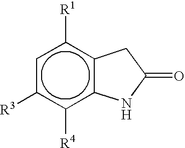

Dialkylation of the indolinone can be performed as described above to prepare a compound of the following structure, wherein R¹-R⁴, and R⁶ are defined above.

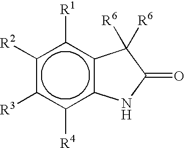

The dialkylated compound is then brominated using a brominating reagent as described in US Patent Application Publication No. US-2006/030717, which is hereby incorporated by reference. A number of brominating reagents are known in the art and include, without limitation, N-bromosuccinimide (NBS), bromine, dibromodimethylhydantoin. In one embodiment, the brominating agent is NBS. In another embodiment, the brominating agent is dibromodimethylhydantoin. In a further embodiment, the brominating agent is bromine. The bromination is performed in any inert solvent that is capable of completely or partially dissolving the indolinone. Examples of suitable solvents include, without limitation, THF, dioxane, acetic acid, acetonitrile, water, dichloroethane, dichloromethane, chlorobenzene, chloroform, or CCl₄. Desirably, the bromination is performed at a temperature of about −20° C. to about 50° C. Most desirably, the bromination is performed at a temperature of about 0° C. to about 25° C. In one embodiment, the bromination may be performed using about 1 equivalent of NBS in MeCN at room temperature.

Finally, the brominated indolinone is then coupled with a pyrrole reagent as described above to prepare the compound of the following structure, wherein R¹, R³, R⁴, R⁶, R⁹, and R¹⁰ are defined above.

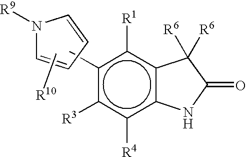

In one embodiment, the process includes preparing a compound of the structure: 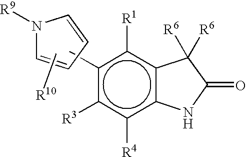
wherein, R¹, R³, and R⁴ are, independently, selected from among H, chlorine, C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, CF₃, SR⁵, OR⁵, N(R⁵)₂, and SO₂N(R⁵)₂; or R¹ and R²; R² and R³; R³ and R⁴; R¹, R², and R³; or R², R³, and R⁴ are fused to form (a) a 3 to 15 membered saturated or unsaturated carbon-containing ring; or (b) a 3 to 15 membered heterocyclic ring containing in its backbone from 1 to 3 heteroatoms selected from the group consisting of O, S, and NR¹¹; and R⁵ is selected from among C₁ to C₆ alkyl and C₁ to C₆ substituted alkyl; R⁶ and R⁷ are the same and are selected from the group consisting of C₁ to C₆ alkyl, substituted C₁ to C₆ alkyl, C₃ to C₁₄ cycloalkyl, substituted and C₃ to C₁₄ cycloalkyl; or R⁶ and R⁷ are fused to form a 3 to 8 membered saturated carbon-containing ring; R⁹ is selected from among C₁ to C₆ alkyl, substituted C₁ to C₆ alkyl, aryl, substituted aryl, heteroaryl, substituted heteroaryl, and COR^{A}; R^{A} is selected from among H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl; R¹⁰ is selected from among H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A}; and R¹¹ is absent, H, C₁ to C₆ alkyl, substituted C₁ to C₆ alkyl, aryl, or substituted aryl; the process including (i) BOC protecting a compound of the structure: 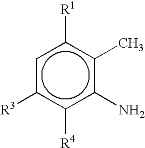
(ii) lithiating the product of step (i); (iii) reacting the product of step (ii) with CO₂ or Y¹C(O)X⁵; wherein, X⁵ and Y¹ are independently chlorine, bromine, C₁ to C₆ alkoxy, or substituted C₁ to C₆ alkoxy; (iv) deprotecting the product of step (iii); (v) cycloamidating the product of step (iv) to form a compound of the structure: 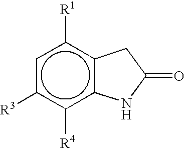
(vi) dialkylating the product of step (v) in the presence of 2 equivalents of a first base, at least 1 equivalent of lithium diisopropylamide, and at least 2 equivalents of an alkylating agent to form a compound of the structure: 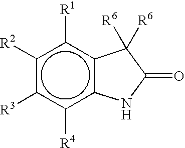
(vii) brominating the product of step (vi) to form a compound of the structure: 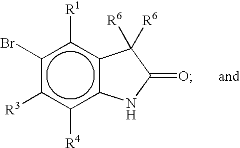
(viii) reacting the product of step (vii) with a compound of the structure:

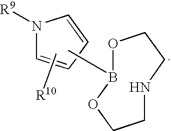

### Processes for Preparing 5-(4′-fluoro-2′-oxospiro[cyclopropane-1,3′-indoline]-5′-yl)-1-methyl-1H-pyrrole-2-carbonitrile and Intermediates Thereof

In another embodiment, a process for preparing 5-(4′-fluoro-2′-oxospiro[cyclopropane-1,3′-indoline]-5′-yl)-1-methyl-1H-pyrrole-2-carbonitrile is described. The process includes (i) BOC protecting a compound of the structure: 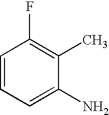
to form a compound of the structure: 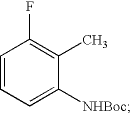
(ii) lithiating the product of step (i) to form a compound of the structure: 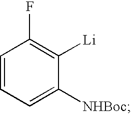
(iii) reacting the product of step (ii) with carbon dioxide to form a compound of the structure: 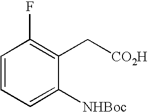
(iv) deprotecting the product of step (iii); (v) cycloamidating the product of step (iv) to form a compound of the structure: 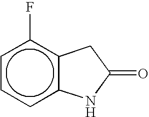
(vi) dialkylating the compound of step (v) using about 3 to about 4 equivalents of lithium diisopropylamide and at least 2 equivalents of 1,2-dibromoethane to form a compound of the structure: 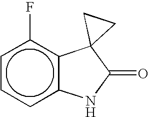
(vii) brominating the product of step (vi) to form a compound of the structure: 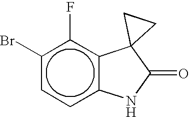
(viii) reacting the product of step (vii) with a compound of the structure:

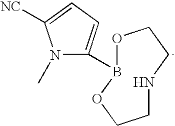

In yet another embodiment, a method of preparing 4′-Fluorospiro[cyclopropane-1,3′-indolin]-2′-one is provided and includes reacting 4-fluoroindolin-2-one and lithium diisopropylamide and thereby adding 1,2-dibromoethane to the product. In one embodiment, the lithium diisopropylamide is prepared by reacting about 2.1 equivalents of diisopropyl amine and 4 equivalents of butyl lithium. Desirably, about 3 equivalents of 1,2-dibromoethane are present In a further embodiment, a process for preparing 5-(4′-fluoro-2′-oxospiro[cyclopropane-1,3′-indoline]-5′-yl)-1-methyl-1H-pyrrole-2-carbonitrile is described in Scheme 4.

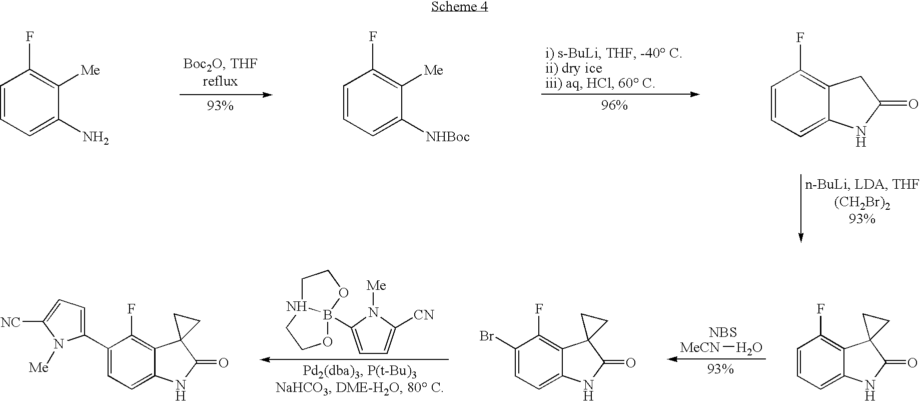

### Spectral Characterization of Polymorph Form A of 5-(4′-fluoro-2′-oxospiro[cyclopropane-1,3′-indoline]-5′-yl)-1-methyl-1H-pyrrole-2-carbonitrile

Characterization of polymorph Form A of 5-(4′-fluoro-2′-oxospiro[cyclopropane-1,3′-indoline]-5′-yl)-1-methyl-1H-pyrrole-2-carbonitrile prepared as described herein may be accomplished using techniques known to those of skill in the art. Specifically, spectral characteristics for polymorph Form A of 5-(4′-fluoro-2′-oxospiro[cyclopropane-1,3′-indoline]-5′-yl)-1-methyl-1H-pyrrole-2-carbonitrile can be performed using techniques including melting point, infrared (IR) spectroscopy, nuclear magnetic resonance (NMR) spectroscopy, mass spectral (MS) analysis, combustion analysis, Raman spectroscopy, elemental analysis, chromatography including high performance liquid chromatography, and microscopy. Other techniques including differential scanning calorimetry (DSC) and X-ray diffraction (XRD) are also useful.

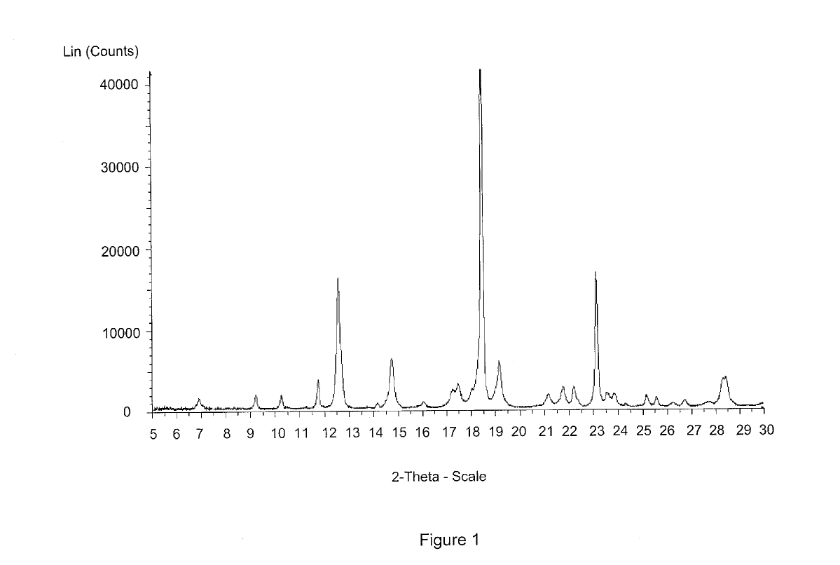

In one embodiment, XRD techniques may be utilized to characterize polymorph Form A of 5-(4′-fluoro-2′-oxospiro[cyclopropane-1,3′-indoline]-5′-yl)-1-methyl-1H-pyrrole-2-carbonitrile. Using the information provided herein, one of skill in the art would readily be able to determine the conditions required to obtain an XRD pattern of polymorph Form A of 5-(4′-fluoro-2′-oxospiro[cyclopropane-1,3′-indoline]-5′-yl)-1-methyl-1H-pyrrole-2-carbonitrile. A variety of XRD instruments are available and include the D8 ADVANCE X-ray powder diffractometer (Bruker), among others. The powder XRD pattern of polymorph Form A of 5-(4′-fluoro-2′-oxospiro[cyclopropane-1,3′-indoline]-5′-yl)-1-methyl-1H-pyrrole-2-carbonitrile was obtained using X-ray crystallographic techniques known to those of skill in the art. See, FIG. 1. In one embodiment, the XRD pattern of polymorph Form A of 5-(4′-fluoro-2′-oxospiro[cyclopropane-1,3′-indoline]-5′-yl)-1-methyl-1H-pyrrole-2-carbonitrile contains one large peak and several smaller peaks. In another embodiment, the XRD for polymorph Form A of 5-(4′-fluoro-2′-oxospiro[cyclopropane-1,3′-indoline]-5′-yl)-1-methyl-1H-pyrrole-2-carbonitrile includes a peak at 2θ of about 18.5°+0.3° at greater than about 95% relative intensity. Desirably, the XRD for polymorph Form A of 5-(4′-fluoro-2′-oxospiro[cyclopropane-1,3′-indoline]-5′-yl)-1-methyl-1H-pyrrole-2-carbonitrile includes a peak at 2θ of about 18.5°+0.3° at greater than about 100% relative intensity. The XRD for polymorph Form A of 5-(4′-fluoro-2′-oxospiro[cyclopropane-1,3′-indoline]-5′-yl)-1-methyl-1H-pyrrole-2-carbonitrile may also include peaks at 2θ of about 14.7° and 19.1°.

DSC techniques can also be utilized to characterize polymorph Form A of 5-(4′-fluoro-2′-oxospiro[cyclopropane-1,3′-indoline]-5′-yl)-1-methyl-1H-pyrrole-2-carbonitrile. One of skill in the art would readily be able to determine the conditions necessary to obtain a DSC thermogram of polymorph Form A of 5-(4′-fluoro-2′-oxospiro[cyclopropane-1,3′-indoline]-5′-yl)-1-methyl-1H-pyrrole-2-carbonitrile. A variety of differential scanning calorimeters are available to those of skill in the art and include the Q series™ DSC Q1000 instrument (TA instruments) using temperatures of about 25° C. to about 230° C. and temperature increases at various rates including 5° C./minute, 10° C./minute, and 30° C./minute, among other instruments and conditions. In one embodiment, the DSC thermogram of polymorph Form A of 5-(4′-fluoro-2′-oxospiro[cyclopropane-1,3′-indoline]-5′-yl)-1-methyl-1H-pyrrole-2-carbonitrile includes an endothermic peak with a Tₒₙₛₑₜ of about 223° C.±1° C.

Solid state nuclear magnetic resonance (NMR) can further be utilized to distinguish characterize polymorph Form A of 5-(4′-fluoro-2′-oxospiro[cyclopropane-1,3′-indoline]-5′-yl)-1-methyl-1H-pyrrole-2-carbonitrile. One of skill in the art would readily be able to determine the conditions necessary to obtain a solid state NMR spectrum of polymorph Form A of 5-(4′-fluoro-2′-oxospiro[cyclopropane-1,3′-indoline]-5′-yl)-1-methyl-1H-pyrrole-2-carbonitrile. A variety of NMR instruments useful for solid state NMR are available to and could readily be selected by those of skill in the art.

Microscopy may also be utilized to determine the crystallographic shape of the particles of polymorph Form A of 5-(4′-fluoro-2′-oxospiro[cyclopropane-1,3′-indoline]-5′-yl)-1-methyl-1H-pyrrole-2-carbonitrile. One of skill in the art would readily be able to select a suitable microscope for such an analysis.

### Compositions Containing Polymorph Form A of 5-(4′-fluoro-2′-oxospiro[cyclopropane-1,3′-indoline]-5′-yl)-1-methyl-1H-pyrrole-2-carbonitrile

Also provided are compositions, desirably pharmaceutical compositions, containing polymorph Form A of 5-(4′-fluoro-2′-oxospiro[cyclopropane-1,3′-indoline]-5′-yl)-1-methyl-1H-pyrrole-2-carbonitrile. In one embodiment, a pharmaceutical composition containing polymorph Form A of 5-(4′-fluoro-2′-oxospiro[cyclopropane-1,3′-indoline]-5′-yl)-1-methyl-1H-pyrrole-2-carbonitrile and a pharmaceutically acceptable carrier is provided. The polymorph Form A of 5-(4′-fluoro-2′-oxospiro[cyclopropane-1,3′-indoline]-5′-yl)-1-methyl-1H-pyrrole-2-carbonitrile samples may be micronized under nitrogen and conventional micronizing techniques, for example with a Trost or jet mill.

The compositions typically contain a pharmaceutically acceptable carrier, but can also contain other suitable components. Typically, the additional components are inert and do not interfere with the function of the required components of the compositions. The compositions can further include other adjuvants, syrups, elixirs, diluents, binders, lubricants, surfactants, granulating agents, disintegrating agents, emollients, metal chelators, pH adjustors, surfactants, fillers, disintegrants, and combinations thereof, among others.

Adjuvants can include, without limitation, flavoring agents, coloring agents, preservatives, and supplemental antioxidants, which can include vitamin E, ascorbic acid, butylated hydroxytoluene (BHT) and butylated hydroxyanisole (BHA).

Binders can include, without limitation, povidone, cellulose, methylcellulose, hydroxymethylcellulose, carboxymethylcellulose calcium, carboxymethylcellulose sodium, hydroxypropylcellulose, hydroxypropylmethylcellulose phthalate, noncrystalline cellulose, polypropylpyrrolidone, polyvinylpyrrolidone (povidone, PVP), gelatin, gum arabic and acacia, polyethylene glycols, starch, sugars such as sucrose, kaolin, dextrose, and lactose, cholesterol, tragacanth, stearic acid, gelatin, casein, lecithin (phosphatides), cetostearyl alcohol, cetyl alcohol, cetyl esters wax, dextrates, dextrin, glyceryl monooleate, glyceryl monostearate, glyceryl palmitostearate, polyoxyethylene alkyl ethers, polyoxyethylene castor oil derivatives, polyoxyethylene stearates, polyvinyl alcohol, and gelatin, among others. In one embodiment, the binder is povidone.

Lubricants can include light anhydrous silicic acid, talc, stearic acid, sodium lauryl sulfate, magnesium stearate and sodium stearyl fumarate, among others. In one embodiment, the lubricant is magnesium stearate.

Granulating agents can include, without limitation, silicon dioxide, starch, calcium carbonate, pectin, crospovidone, and polyplasdone, among others.

Disintegrating agents or disintegrants can include starch, carboxymethylcellulose, substituted hydroxypropylcellulose, sodium bicarbonate, calcium phosphate, calcium citrate, sodium starch glycolate, pregelatinized starch or crospovidone, among others.

Emollients can include, without limitation, stearyl alcohol, mink oil, cetyl alcohol, oleyl alcohol, isopropyl laurate, polyethylene glycol, olive oil, petroleum jelly, palmitic acid, oleic acid, and myristyl myristate.

Surfactants can include polysorbates, sorbitan esters, poloxamer, or sodium lauryl sulfate. In one embodiment, the surfactant is sodium lauryl sulfate.

Metal chelators can include physiologically acceptable chelating agents including edetic acid, malic acid, or fumaric acid. In one embodiment, the metal chelator is edetic acid.

pH adjusters can also be utilized to adjust the pH of a solution containing polymorph Form A of 5-(4′-fluoro-2′-oxospiro[cyclopropane-1,3′-indoline]-5′-yl)-1-methyl-1H-pyrrole-2-carbonitrile to about 4, about 5, or about 6. In one embodiment, the pH of a solution containing polymorph Form A of 5-(4′-fluoro-2′-oxospiro[cyclopropane-1,3′-indoline]-5′-yl)-1-methyl-1H-pyrrole-2-carbonitrile is adjusted to a pH of about 4.6. pH adjustors can include physiologically acceptable agents including citric acid, ascorbic acid, fumaric acid, or malic acid, and salts thereof. In one embodiment, the pH adjuster is citric acid.

Additional fillers that can be used in the composition include mannitol, calcium phosphate, pregelatinized starch, or sucrose.

### Methods of Using Polymorph Form A of 5-(4′-fluoro-2′-oxospiro[cyclopropane-1,3′-indoline]-5′-yl)-1-methyl-1H-pyrrole-2-carbonitrile

Further provided are methods of delivering polymorph Form A of 5-(4′-fluoro-2′-oxospiro[cyclopropane-1,3′-indoline]-5′-yl)-1-methyl-1H-pyrrole-2-carbonitrile to a patient.

The dosage requirements of polymorph Form A of polymorph Form A of 5-(4′-fluoro-2′-oxospiro[cyclopropane-1,3′-indoline]-5′-yl)-1-methyl-1H-pyrrole-2-carbonitrile may vary based on the severity of the symptoms presented and the particular subject being treated. Treatment can be initiated with small dosages less than the optimum dose of polymorph Form A of 5-(4′-fluoro-2′-oxospiro[cyclopropane-1,3′-indoline]-5′-yl)-1-methyl-1H-pyrrole-2-carbonitrile. Thereafter the dosage is increased until the optimum effect under the circumstances is reached. Precise dosages will be determined by the administering physician based on experience with the individual subject treated. In general, polymorph Form A of 5-(4′-fluoro-2′-oxospiro[cyclopropane-1,3′-indoline]-5′-yl)-1-methyl-1H-pyrrole-2-carbonitrile is most desirably administered at a concentration that will generally afford effective results without causing any unacceptable harmful or deleterious side effects. For example, an effective amount of polymorph Form A of 5-(4′-fluoro-2′-oxospiro[cyclopropane-1,3′-indoline]-5′-yl)-1-methyl-1H-pyrrole-2-carbonitrile is generally, e.g., about 0.05 mg to about 1 mg, about 0.05 mg to about 0.3 mg, about 0.05 mg, about 0.075 mg, about 0.1 mg, about 0.15 mg, about 0.2 mg, or about 0.3 mg.

Polymorph Form A of 5-(4′-fluoro-2′-oxospiro[cyclopropane-1,3′-indoline]-5′-yl)-1-methyl-1H-pyrrole-2-carbonitrile is therefore useful in contraception and hormone replacement therapy. Polymorph Form A of 5-(4′-fluoro-2′-oxospiro[cyclopropane-1,3′-indoline]-5′-yl)-1-methyl-1H-pyrrole-2-carbonitrile is also useful in the treatment and/or prevention of fibroids, specifically uterine fibroids; benign prostatic hypertrophy; benign and malignant neoplastic disease; dysfunctional bleeding; uterine leiomyomata; endometriosis; polycystic ovary syndrome; and hormone-dependent carcinomas and adenocarcinomas of the pituitary, endometrium, kidney, uterine, ovary, breast, colon, and prostate and other hormone-dependent tumors; and treating symptoms of premenstrual syndrome and premenstrual dysphoric disorder. Additional uses of polymorph Form A of 5-(4′-fluoro-2′-oxospiro[cyclopropane-1,3′-indoline]-5′-yl)-1-methyl-1H-pyrrole-2-carbonitrile include stimulation of food intake or the synchronization of estrus.

Polymorph Form A of 5-(4′-fluoro-2′-oxospiro[cyclopropane-1,3′-indoline]-5′-yl)-1-methyl-1H-pyrrole-2-carbonitrile can be formulated in any form suitable for the desired route of delivery using a pharmaceutically effective amount of polymorph Form A of 5-(4′-fluoro-2′-oxospiro[cyclopropane-1,3′-indoline]-5′-yl)-1-methyl-1H-pyrrole-2-carbonitrile. For example, polymorph Form A of 5-(4′-fluoro-2′-oxospiro[cyclopropane-1,3′-indoline]-5′-yl)-1-methyl-1H-pyrrole-2-carbonitrile can be delivered by a route such as oral, dermal, transdermal, intrabronchial, intranasal, intravenous, intramuscular, subcutaneous, parenteral, intraperitoneal, intranasal, vaginal, rectal, sublingual, intracranial, epidural, intratracheal, or by sustained release. Desirably, delivery is oral.

For example, polymorph Form A of 5-(4′-fluoro-2′-oxospiro[cyclopropane-1,3′-indoline]-5′-yl)-1-methyl-1H-pyrrole-2-carbonitrile may be formulated for administration orally in such forms as tablets, capsules, microcapsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like. The preferred pharmaceutical compositions from the standpoint of ease of preparation and administration are solid compositions, particularly tablets and hard-filled or liquid-filled capsules.

Polymorph Form A of 5-(4′-fluoro-2′-oxospiro[cyclopropane-1,3′-indoline]-5′-yl)-1-methyl-1H-pyrrole-2-carbonitrile may also be administered parenterally or intraperitoneally. Solutions or suspensions of polymorph Form A of 5-(4′-fluoro-2′-oxospiro[cyclopropane-1,3′-indoline]-5′-yl)-1-methyl-1H-pyrrole-2-carbonitrile can be prepared in water suitably mixed with a surfactant such as hydroxypropylcellulose. Dispersions can also be prepared in glycerol, liquid, polyethylene glycols and mixtures thereof in oils. Under ordinary conditions of storage and use, these preparations contain a preservative to prevent the growth of microorganisms. Typically, such sterile injectable solutions or suspensions contain from about 0.05 to 5% suspending agent in an isotonic medium. Such pharmaceutical preparations may contain, for example, from about 25 to about 90% of the active ingredient in combination with the carrier, more usually between about 5% and 60% by weight.

In another embodiment, polymorph Form A of 5-(4′-fluoro-2′-oxospiro[cyclopropane-1,3′-indoline]-5′-yl)-1-methyl-1H-pyrrole-2-carbonitrile is delivered intravenously, intramuscularly, subcutaneously, parenterally and intraperitoneally in the form of sterile injectable solutions, suspensions, dispersions, and powders which are fluid to the extent that easy syringe ability exits. Such injectable compositions are sterile, stable under conditions of manufacture and storage, and free of the contaminating action of microorganisms such as bacteria and fungi.

The carrier can be a solvent or dispersion medium containing, for example, water, ethanol (e.g., glycerol, propylene glycol and liquid polyethylene glycol), oils, and mixtures thereof. Desirably the liquid carrier is water. In one embodiment, the oil is vegetable oil. Optionally, the liquid carrier contains a suspending agent. In another embodiment, the liquid carrier is an isotonic medium and contains 0.05 to about 5% suspending agent.

In a further embodiment, polymorph Form A of 5-(4′-fluoro-2′-oxospiro[cyclopropane-1,3′-indoline]-5′-yl)-1-methyl-1H-pyrrole-2-carbonitrile is delivered rectally in the form of a conventional suppository.

In another embodiment, polymorph Form A of 5-(4′-fluoro-2′-oxospiro[cyclopropane-1,3′-indoline]-5′-yl)-1-methyl-1H-pyrrole-2-carbonitrile is delivered vaginally in the form of a conventional suppository, cream, gel, ring, or coated intrauterine device (IUD).

In yet another embodiment, polymorph Form A of 5-(4′-fluoro-2′-oxospiro[cyclopropane-1,3′-indoline]-5′-yl)-1-methyl-1H-pyrrole-2-carbonitrile is delivered intranasally or intrabronchially in the form of an aerosol.

In a further embodiment, polymorph Form A of 5-(4′-fluoro-2′-oxospiro[cyclopropane-1,3′-indoline]-5′-yl)-1-methyl-1H-pyrrole-2-carbonitrile is delivered transdermally or by sustained release through the use of a transdermal patch containing polymorph Form A of 5-(4′-fluoro-2′-oxospiro[cyclopropane-1,3′-indoline]-5′-yl)-1-methyl-1H-pyrrole-2-carbonitrile and an optional carrier that is inert to polymorph Form A of 5-(4′-fluoro-2′-oxospiro[cyclopropane-1,3′-indoline]-5′-yl)-1-methyl-1H-pyrrole-2-carbonitrile, is nontoxic to the skin, and allows for delivery of polymorph Form A of 5-(4′-fluoro-2′-oxospiro[cyclopropane-1,3′-indoline]-5′-yl)-1-methyl-1H-pyrrole-2-carbonitrile for systemic absorption into the blood stream. Such a carrier can be a cream, ointment, paste, gel, or occlusive device. The creams and ointments can be viscous liquid or semisolid emulsions. Pastes include absorptive powders dispersed in petroleum or hydrophilic petroleum. Further, a variety of occlusive devices can be utilized to release polymorph Form A of 5-(4′-fluoro-2′-oxospiro[cyclopropane-1,3′-indoline]-5′-yl)-1-methyl-1H-pyrrole-2-carbonitrile into the blood stream and include semi-permeable membranes covering a reservoir contain the active reagents, or a matrix containing the reactive reagents.

The use of sustained delivery devices can be desirable, in order to avoid the necessity for the patient to take medications on a daily basis. The term “sustained delivery” is used herein to refer to delaying the release of an active agent, i.e., polymorph Form A of 5-(4′-fluoro-2′-oxospiro[cyclopropane-1,3′-indoline]-5′-yl)-1-methyl-1H-pyrrole-2-carbonitrile, until after placement in a delivery environment, followed by a sustained release of the agent at a later time. A number of sustained delivery devices are known in the art and include hydrogels (U.S. Pat. Nos. 5,266,325; 4,959,217; 5,292,515), osmotic pumps (U.S. Pat. Nos. 4,295,987 and 5,273,752 and European Patent No. 314,206, among others); hydrophobic membrane materials, such as ethylenemethacrylate (EMA) and ethylenevinylacetate (EVA); bioresorbable polymer systems (International Patent Publication No. WO 98/44964 and U.S. Pat. Nos. 5,756,127 and 5,854,388); and other bioresorbable implant devices composed of, for example, polyesters, polyanhydrides, or lactic acid/glycolic acid copolymers (U.S. Pat. No. 5,817,343). For use in such sustained delivery devices, polymorph Form A of 5-(4′-fluoro-2′-oxospiro[cyclopropane-1,3′-indoline]-5′-yl)-1-methyl-1H-pyrrole-2-carbonitrile can be formulated as described herein. See, U.S. Pat. Nos. 3,845,770; 3,916,899; 3,536,809; 3,598,123; and 4,008,719.

Desirably, polymorph Form A of 5-(4′-fluoro-2′-oxospiro[cyclopropane-1,3′-indoline]-5′-yl)-1-methyl-1H-pyrrole-2-carbonitrile is formed into a suitable dosing unit for delivery to a patient. Suitable dosing units include oral dosing units, such as a directly compressible tablets, capsules, powders, suspensions, microcapsules, dispersible powders, granules, suspensions, syrups, elixirs, and aerosols. In one embodiment, polymorph Form A of 5-(4′-fluoro-2′-oxospiro[cyclopropane-1,3′-indoline]-5′-yl)-1-methyl-1H-pyrrole-2-carbonitrile is compressed into a tablet, which is optionally added to a capsule, or polymorph Form A of 5-(4′-fluoro-2′-oxospiro [cyclopropane-1,3′-indoline]-5′-yl)-1-methyl-1H-pyrrole-2-carbonitrile is added directly to a capsule. Polymorph Form A of 5-(4′-fluoro-2′-oxospiro[cyclopropane-1,3′-indoline]-5′-yl)-1-methyl-1H-pyrrole-2-carbonitrile can also be formulated for delivery by other suitable routes. These dosing units are readily prepared using the methods described herein and those known to those of skill in the art.

Solid forms, including tablets, caplets, and capsules containing polymorph Form A of 5-(4′-fluoro-2′-oxospiro[cyclopropane-1,3′-indoline]-5′-yl)-1-methyl-1H-pyrrole-2-carbonitrile can be formed by dry blending polymorph Form A of 5-(4′-fluoro-2′-oxospiro[cyclopropane-1,3′-indoline]-5′-yl)-1-methyl-1H-pyrrole-2-carbonitrile with the components described above. In one embodiment, the capsules utilized include hydroxypropyl methylcellulose, hypromellose capsule, or a hard shell gelatin capsule. The tablets or caplets that contain polymorph Form A of 5-(4′-fluoro-2′-oxospiro[cyclopropane-1,3′-indoline]-5′-yl)-1-methyl-1H-pyrrole-2-carbonitrile are optionally film-coated. Suitable film-coatings are known to those of skill in the art. For example, the film-coating can be selected from among polymers such as hydroxypropylmethylcellulose, ethyl cellulose, polyvinyl alcohol, and combinations thereof.

A pharmaceutically effective amount of polymorph Form A of 5-(4′-fluoro-2′-oxospiro[cyclopropane-1,3′-indoline]-5′-yl)-1-methyl-1H-pyrrole-2-carbonitrile can vary depending on the other components of the composition being delivered, mode of delivery, severity of the condition being treated, the patient's age and weight, and any other active ingredients used in the composition. The dosing regimen can also be adjusted to provide the optimal therapeutic response. Several divided doses can be delivered daily, e.g., in divided doses 2 to 4 times a day, or a single dose can be delivered. The dose can however be proportionally reduced or increased as indicated by the exigencies of the therapeutic situation. In one embodiment, the delivery is on a daily, weekly, or monthly basis. In another embodiment, the delivery is on a daily delivery. However, daily dosages can be lowered or raised based on the periodic delivery.

It is contemplated that when polymorph Form A of 5-(4′-fluoro-2′-oxospiro[cyclopropane-1,3′-indoline]-5′-yl)-1-methyl-1H-pyrrole-2-carbonitrile is used for contraception or hormone replacement therapy, it can be administered in conjunction with one or more other progesterone receptor agonists, estrogen receptor agonists, progesterone receptor antagonists, and selective estrogen receptor modulators, among others.

When utilized for treating neoplastic disease, carcinomas, and adenocarcinomas, polymorph Form A of 5-(4′-fluoro-2′-oxospiro[cyclopropane-1,3′-indoline]-5′-yl)-1-methyl-1H-pyrrole-2-carbonitrile can be administered in conjunction with one or more chemotherapeutic agents which can readily be selected by one of skill in the art.

In one embodiment, a method of preparing a pharmaceutical composition containing polymorph Form A of 5-(4′-fluoro-2′-oxospiro[cyclopropane-1,3′-indoline]-5′-yl)-1-methyl-1H-pyrrole-2-carbonitrile is described and includes combining polymorph Form A of 5-(4′-fluoro-2′-oxospiro[cyclopropane-1,3′-indoline]-5′-yl)-1-methyl-1H-pyrrole-2-carbonitrile and one or more of a metal chelator, a pH adjuster, a surfactant, at least one filler, a binder, a disintegrant, and a lubricant.

### Kits Containing Polymorph Form A of 5-(4′-fluoro-2′-oxospiro[cyclopropane-1,3′-indoline]-5′-yl)-1-methyl-1H-pyrrole-2-carbonitrile

Also provided are kits or packages containing polymorph Form A of 5-(4′-fluoro-2′-oxospiro[cyclopropane-1,3′-indoline]-5′-yl)-1-methyl-1H-pyrrole-2-carbonitrile prepared as described herein. Kits can include polymorph Form A of 5-(4′-fluoro-2′-oxospiro[cyclopropane-1,3′-indoline]-5′-yl)-1-methyl-1H-pyrrole-2-carbonitrile and a carrier suitable for administration to a mammalian subject as discussed above. Typically, the tablets or capsules are packaged in blister packs, and desirably Ultrx™ 2000 blister packs. In one embodiment, a kit is provided and contains polymorph Form A of 5-(4′-fluoro-2′-oxospiro[cyclopropane-1,3′-indoline]-5′-yl)-1-methyl-1H-pyrrole-2-carbonitrile; and a carrier suitable for administration to a mammalian subject is described.

The kits or packages containing polymorph Form A of 5-(4′-fluoro-2′-oxospiro[cyclopropane-1,3′-indoline]-5′-yl)-1-methyl-1H-pyrrole-2-carbonitrile are designed for use in the regimens described herein. These kits are desirably designed for daily oral delivery over 21-day, 28-day, 30-day, or 31-day cycles, among others, and more desirably for one oral delivery per day. When polymorph Form A of 5-(4′-fluoro-2′-oxospiro[cyclopropane-1,3′-indoline]-5′-yl)-1-methyl-1H-pyrrole-2-carbonitrile is to be delivered continuously, a package or kit can include polymorph Form A of 5-(4′-fluoro-2′-oxospiro[cyclopropane-1,3′-indoline]-5′-yl)-1-methyl-1H-pyrrole-2-carbonitrile in each tablet. When polymorph Form A of 5-(4′-fluoro-2′-oxospiro[cyclopropane-1,3′-indoline]-5′-yl)-1-methyl-1H-pyrrole-2-carbonitrile is to be delivered with periodic discontinuation, a package or kit can include placebos on those days when polymorph Form A of 5-(4′-fluoro-2′-oxospiro[cyclopropane-1,3′-indoline]-5′-yl)-1-methyl-1H-pyrrole-2-carbonitrile is not delivered.

Additional components may be co-administered with polymorph Form A of 5-(4′-fluoro-2′-oxospiro[cyclopropane-1,3′-indoline]-5′-yl)-1-methyl-1H-pyrrole-2-carbonitrile and include progestational agents, estrogens, and selective estrogen receptor modulators.

The kits are also desirably organized to indicate a single oral formulation or combination of oral formulations to be taken on each day of the cycle, desirably including oral tablets to be taken on each of the days specified, and more desirably one oral tablet will contain each of the combined daily dosages indicated.

In one embodiment, a kit can include a single phase of a daily dosage of polymorph Form A of 5-(4′-fluoro-2′-oxospiro[cyclopropane-1,3′-indoline]-5′-yl)-1-methyl-1H-pyrrole-2-carbonitrile over a 21-day, 28-day, 30-day, or 31-day cycle. Alternatively, a kit can include a single phase of a daily dosage of polymorph Form A of 5-(4′-fluoro-2′-oxospiro[cyclopropane-1,3′-indoline]-5′-yl)-1-methyl-1H-pyrrole-2-carbonitrile over the first 21 days of a 28-day, 30-day, or 31-day cycle. A kit can also include a single phase of a daily dosage of polymorph Form A of 5-(4′-fluoro-2′-oxospiro[cyclopropane-1,3′-indoline]-5′-yl)-1-methyl-1H-pyrrole-2-carbonitrile over the first 28 days of a 30-day or 31-day cycle.

In a further embodiment, a kit can include a single combined phase of a daily dosage of polymorph Form A of 5-(4′-fluoro-2′-oxospiro[cyclopropane-1,3′-indoline]-5′-yl)-1-methyl-1H-pyrrole-2-carbonitrile and a progestational agent over a 21-day, 28-day, 30-day, or 31-day cycle. Alternatively, a kit can include a single combined phase of a daily dosage of polymorph Form A of 5-(4′-fluoro-2′-oxospiro[cyclopropane-1,3′-indoline]-5′-yl)-1-methyl-1H-pyrrole-2-carbonitrile and a progestational agent over the first 21 days of a 28-day, 30-day, or 31-day cycle. A kit can also include a single combined phase of a daily dosage of polymorph Form A of 5-(4′-fluoro-2′-oxospiro[cyclopropane-1,3′-indoline]-5′-yl)-1-methyl-1H-pyrrole-2-carbonitrile and a progestational agent over the first 28 days of a 30-day or 31-day cycle.

In another embodiment, a 28-day kit can include a first phase of from 14 to 28 daily dosage units of polymorph Form A of 5-(4′-fluoro-2′-oxospiro[cyclopropane-1,3′-indoline]-5′-yl)-1-methyl-1H-pyrrole-2-carbonitrile; a second phase of from 1 to 11 daily dosage units of a progestational agent; and, optionally, a third phase of an orally and pharmaceutically acceptable placebo for the remaining days of the cycle.

In yet a further embodiment, a 28-day kit can include a first phase of from 14 to 21 daily dosage units of polymorph Form A of 5-(4′-fluoro-2′-oxospiro[cyclopropane-1,3′-indoline]-5′-yl)-1-methyl-1H-pyrrole-2-carbonitrile; a second phase of from 1 to 11 daily dosage units of a progestational agent; and, optionally, a third phase of an orally and pharmaceutically acceptable placebo for the remaining days of the cycle.

In another embodiment, a 28-day kit can include a first phase of from 18 to 21 daily dosage units of polymorph Form A of 5-(4′-fluoro-2′-oxospiro[cyclopropane-1,3′-indoline]-5′-yl)-1-methyl-1H-pyrrole-2-carbonitrile; a second phase of from 1 to 7 daily dose units of a progestational agent; and, optionally, an orally and pharmaceutically acceptable placebo for each of the remaining 0 to 9 days in the 28-day cycle.

In yet a further embodiment, a 28-day kit can include a first phase of 21 daily dosage units of polymorph Form A of 5-(4′-fluoro-2′-oxospiro[cyclopropane-1,3′-indoline]-5′-yl)-1-methyl-1H-pyrrole-2-carbonitrile; a second phase of 3 daily dosage units for days 22 to 24 of a progestational agent; and, optionally, a third phase of 4 daily units of an orally and pharmaceutically acceptable placebo for each of days 25 to 28.

In another embodiment, a 28-day kit can include a first phase of from 14 to 21 daily dosage units of a progestational agent equal in progestational activity to about 35 to about 150 μg levonorgestrel, a second phase of from 1 to 11 daily dosage units of polymorph Form A of 5-(4′-fluoro-2′-oxospiro[cyclopropane-1,3′-indoline]-5′-yl)-1-methyl-1H-pyrrole-2-carbonitrile; and optionally, a third phase of an orally and pharmaceutically acceptable placebo for the remaining days of the cycle in which no antiprogestin, progestin or estrogen is administered.

In a further embodiment, a 28-day kit can include a first phase of from 14 to 21 daily dosage units of a progestational agent equal in progestational activity to about 35 to about 100 μg levonorgestrel; a second phase of from 1 to 11 daily dosage units of polymorph Form A of 5-(4′-fluoro-2′-oxospiro[cyclopropane-1,3′-indoline]-5′-yl)-1-methyl-1H-pyrrole-2-carbonitrile; and optionally, a third phase of an orally and pharmaceutically acceptable placebo for the remaining days of the cycle in which no antiprogestin, progestin or estrogen is administered.

Desirably, the daily dosage of polymorph Form A of 5-(4′-fluoro-2′-oxospiro[cyclopropane-1,3′-indoline]-5′-yl)-1-methyl-1H-pyrrole-2-carbonitrile remains fixed in each particular phase in which it is delivered. It is further preferable that the daily dose units described are to be delivered in the order described, with the first phase followed in order by the second and third phases. To help facilitate compliance with each regimen, it is also preferred that the kits contain the placebo described for the final days of the cycle.

A number of packages or kits are known in the art for the use in dispensing pharmaceutical agents for oral use. Desirably, the package has indicators for each day of the 28-day cycle, and more desirably is a labeled blister package, dial dispenser package, or bottle.

The kit can further contain instructions for administering polymorph Form A of 5-(4′-fluoro-2′-oxospiro[cyclopropane-1,3′-indoline]-5′-yl)-1-methyl-1H-pyrrole-2-carbonitrile.

The following examples are illustrative only and are not intended to be a limitation on the present invention.

### EXAMPLES

Nuclear Magnetic Resonance (NMR) spectra of the intermediates were recorded on a Bruker Advance™ DPX300 or DRX400 NMR spectrometer. Spectra were referenced by an internal standard.

High Performance Liquid Chromatography (HPLC) analysis of the intermediates and reaction monitoring was carried out on an Agilent™ 1090 liquid chromatograph equipped with a diode array detector. HPLC analysis of the purity of the final compound was done on an Agilent™ 1100 series chromatograph equipped with a Prodigy™ ODS3, 0.46×15 cm column. Standard method: 90:10 to 10:90 20 min gradient of water-acetonitrile containing 0.02% TFA, flow rate 1 mL/min.

Gas Chromatography (GC) monitoring of the cyclopropanation step was carried out on an Agilent™ GC spectrometer equipped with an FID detector.

LCMS data were obtained on an Agilent™ 1100 LC system with an Agilent™ 1100 LC/MS detector equipped with a CAPCELL PAK C-18 4.6×50 mm column. Standard method: 90:10 to 10:90 8 min gradient of water-acetonitrile containing 0.02% HCO₂H, flow rate 1 mL/min.

Melting points were measured on a Büchi Melting point apparatus and are uncorrected.

### Example 1

### tert-Butyl 3-fluoro-2-methylphenylcarbamate

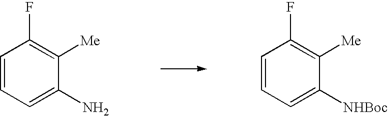

A 2-L round bottom flask, equipped with a magnetic stirrer and a reflux condenser and set in a heating mantle, was charged with 3-fluoro-2-methylaniline (99.1 g, 0.793 mol), di-tert-butyl dicarbonate (190.2 g, 0.872 mol, 1.1 eq) and THF (500 mL). The solution was heated at reflux for 16 hours. The completeness of the reaction was monitored by HPLC (see analytical method below). The reaction mixture was allowed to cool to room temperature and then the solution was concentrated in vacuo (bath temp. 37° C.). The oily residue (255 g) was mixed with heptane (250 mL) which caused slow crystallization of the product. More solvent was removed in vacuum (about 150 mL), thereby resulting in a thick slurry. The slurry was further diluted with 250 mL of heptane and the first crop of crystals was filtered, washed with small amount of cold heptane and dried in air on the filter (109.4 g as white crystals, purity>99.9%).

The filtrate was concentrated in vacuum to about 100 mL and the second crop of crystals was collected by filtration, washed with heptane and dried in air (42.0 g).

The filtrate was further concentrated (about 70 mL) and then chilled in an ice bath. The third crop of crystals was isolated the same way (14.9 g, purity 99%). Total yield of product was 166.3 g (93% theor.).

LCMS (ES+), m/z: 226 (M+H⁺).

HPLC analytical method: Prodigy™ ODS3 4.6×50 cm column, 1 mL/min flow rate, gradient 10 to 90% over 9 min of MeCN-water containing 0.02% of TFA. Retention times: tert-Butyl 3-fluoro-2-methylphenylcarbamate (6.88′), 3-fluoro-2-methylaniline (2.63′).

### Example 2

### 4-Fluoroindolin-2-one

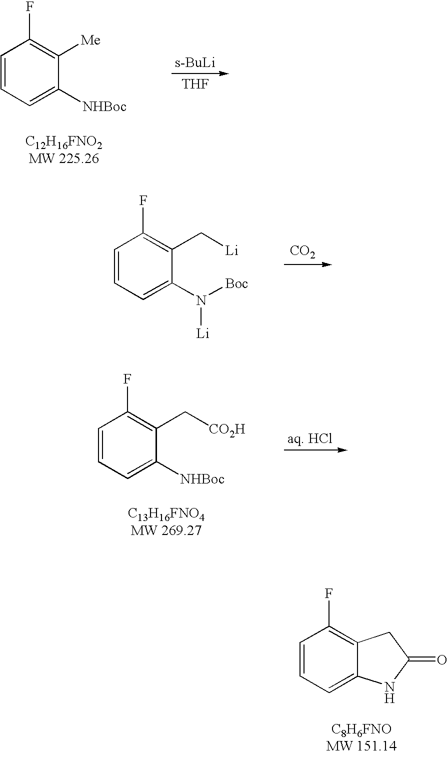

A 5-L round bottom flask equipped with a 1-L addition funnel capped with a rubber septum, a thermocouple, a nitrogen inlet, a mechanical stirrer and a rubber septum was set in an acetone bath cooled with a submersible chiller. The reaction flask was charged with N-Boc-3-fluoro-2-methylaniline (160 g, 0.710 mol) and then was purged with nitrogen. Anhydrous THF (1.6 L) was added, thereby resulting in a clear solution. The solution was chilled to below −45° C. by setting the bath temperature at −60 to −65° C. A 1.4 M solution of sec-BuLi in cyclohexane (1.12 L, 1.56 mol, 2.2 eq.) was transferred to the addition funnel via a cannula and was then slowly added to the solution in the flask at a rate to maintain the temperature during the addition below −40° C. The solution stayed colorless during the addition of the first equivalent of sec-BuLi and then turned bright-yellow at the point where addition of the second equivalent began. The average rate of addition was 11-12 mL/min. When the addition was complete, the reaction mixture was allowed to warm slowly (over a period of 1-1½ hr) to −15 to −20° C. by setting the bath thermostat to chiller's thermostat −15° C. The thermostat was then reset to −60° C. and the reaction mixture was cooled back to −40 to −45° C.

A separate round bottom 5-L flask was set up and equipped with a mechanical stirrer, a rubber septum, a nitrogen inlet and one neck open left open to air. The flask was charged with about 250 g of dry ice and 500 mL of dry THF under a gentle sweep of nitrogen. The solution of the lithiated derivative was slowly transferred into the dry ice slurry via a 12-gauge cannula by applying a slight nitrogen pressure to the first flask. The transfer was completed in about 50 minutes. Additional dry ice was added to the mixture throughout the addition (about 200 g) to ensure saturation of the mixture with carbon dioxide. When the addition was complete, the contents of the flask were allowed to warm to room temperature. HPLC analysis of the mixture showed content of the carboxylic acid at 96 area %, while the amount of the starting aniline was undetectable.

Aqueous HCl (2 M) was carefully added to the reaction mixture containing the carboxylic acid until the pH was about 2 to 3 (about 1.0 L). The biphasic mixture was stirred rapidly for 5-10 min. The aqueous layer was separated and extracted with MTBE (2×500 mL). The extracts were combined with the organic layer and the solution was evaporated to dryness. The residue that remained after evaporation was suspended in water (930 mL) and the slurry was transferred into a 3-L flask equipped with a mechanical stirrer, a reflux condenser, a thermocouple and set in heating mantle. Concentrated aqueous HCl (470 mL, 5.6 mol) was added to the suspension. The reaction mixture was heated at 60° C. overnight (16-20 hr), while the reaction mixture remained heterogeneous throughout the course of the reaction. The reaction was determined to be complete as evidenced by HPLC analysis of the reaction mixture in which 97 area % of the cyclized product and non-detectable starting material was observed. The slurry was cooled to room temperature, the solid was filtered, washed with dilute aqueous NaHCO₃ solution and then water until neutral pH, dried on the filter in a stream of air, and then dried in a vacuum desiccator over CaSO₄. Yield 98.2 g (92%) as an off-white crystalline solid.

M.p. 197-199° C.

¹H NMR (300 MHz, CDCl₃): δ 8.85 (br s, 1H), 7.21 (m, 1H), 6.78-6.68 (m, 2H), 3.58 (s, 2H).

HPLC analytical method: Prodigy™ ODS3 4.6×50 cm column, 1 mL/min flow rate, gradient 10 to 90% over 9 min of MeCN-water containing 0.02% of TFA. Retention times: tert-Butyl 3-fluoro-2-methylphenylcarbamate (5.25′), 4-fluoroindolinone (3.49′), and N-Boc-3-fluoro-2-methylaniline (6.85′).

### Example 3

### 4′-Fluorospiro[cyclopropane-1,3′-indolin]-2′-one

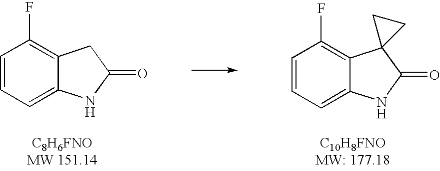

A 2-L round bottom flask equipped with a mechanical stirrer, a 500-mL addition funnel capped with a rubber septum, a nitrogen inlet, a thermocouple, and set in a removable dry ice-acetone bath was purged with nitrogen and charged with 4-fluoroindolin-2-one (30.00 g, 198.5 mmol), diisopropylamine (42.10 g, 58.5 mL, 416.8 mmol, 2.1 eq) and dry THF (400 mL). The contents of the flask were chilled to −20 to −30° C. by adjusting the bath temperature to −40° C. The starting indolinone only partially dissolved in THF at room temperature and re-precipitated as the reaction mixture cooled. A solution of n-BuLi (318 mL, 794 mmol, 4.0 eq) was transferred into the addition funnel via a cannula and then was added to the reaction mixture. The rate of addition was adjusted to keep the temperature of the reaction mixture below −20° C., i.e., at a rate of about 6-7 mL/min. When the addition was complete, the dry ice bath was removed and replaced with regular ice bath. The 500-mL addition funnel was replaced with a clean 125-mL addition funnel. 1,2-Dibromoethane (112.0 g, 51.4 mL, 595.5 mmol, 3.0 eq) was mixed with 50 mL of THF and the mixture was placed into the addition funnel. When the temperature of the reaction mixture reached 0° C., the dibromoethane solution was carefully added to the reaction mixture, maintaining the reaction mixture temperature below about 10° C. The ice bath was then removed and the reaction mixture was left stirring at room temperature. The progress of reaction was monitored by GC. Typically, the reaction was complete after 15-18 hours. After disappearance of the starting material, the reaction mixture was quenched by adding carefully a solution containing brine (40 mL), concentrated aqueous HCl (42 mL) and water (180 mL). The pH of the aqueous layer was about 2 to 3. The phases were then separated, the aqueous layer extracted with MTBE (100 mL) and then combined with the organic phase. The latter was washed with saturated aq. NaHCO₃ solution (50 mL) and filtered through a pad of Silica gel (ø8.5×2 cm high). The filtrate was evaporated in vacuum to dryness and the residue was triturated with heptane (30 mL) and water (50 mL). The solid was filtered, washed with water and heptane and dried in a stream of air on the filter. Yield (crude) 32.6 g (93%).

M.p. 169-73° C.

¹H NMR (DMSO-d₆, 300 MHz): δ 10.80 (br s, 1H), 7.19 (ddd, J=5.7, 7.9, 8.3 Hz, 1H), 6.79 (d, J=7.9 Hz, 1H), 6.74 (dd, J=8.3, 10.2 Hz, 1H), 1.76 (m, 2H), 1.46 (m, 2H).

HPLC analytical method: Prodigy™ ODS3 4.6×50 cm column, 1 mL/min flow rate, gradient 10 to 90% over 9 min of MeCN-water containing 0.02% of TFA. Retention times: 4′-Fluorospiro[cyclopropane-1,3′-indolin]-2′-one (6.88′), 4-fluoroindolin-2-one (2.63′).

GC analytical method: Varian CP-SIL 8 CB low bleed/ms 25 m×250 μm column (part No CP584015), Helium constant flow 1.5 mL/min, temp. ramp 40 to 250° C. at 15° C./min, initial time 0, final time 1 min.

### Example 4

### 5′-Bromo-4′-fluorospiro[cyclopropane-1,3′-indolin]-2′-one

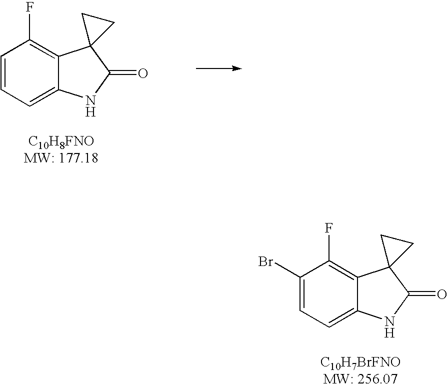

A 1-L round bottom flask equipped with a mechanical stirrer, an addition funnel and a thermocouple, set in an ice bath, was charged with 4′-fluorospiro[cyclopropane-1,3′-indolin]-2′-one (32.0 g, 181 mmol), acetonitrile (200 mL) and water (40 mL). The resulting suspension was chilled to 0° C. N-Bromosuccinimide (32.2 g, 181 mmol) was dissolved in a mixture of acetonitrile (200 mL) and water (40 mL), the solution was placed into the addition funnel and then was slowly added, over about 30 minutes, to the reaction mixture. The resulting mixture was left stirring in the ice bath, thereby gradually reaching room temperature while the course of the reaction was monitored by HPLC to provide the results as set forth in Table 1.

TABLE 1

Area (%) at 215 nm

| | | Starting | p-Br | o-Br | o,p-Br₂ |
|---|---|---|---|---|---|
| Time | | Material | Product | Side-Product | Side-Product |
| 10 | min | 22 | 74 | 1.9 | 0.5 |
| 2.5 | hours | 1.5 | 93 | 1.7 | 0.4 |
| 4 | hours | 3.8 | 93 | 1.6 | 0.6 |
| 18 | hours | 2.4 | 92 | 0.5 | 1.0 |

The crystalline precipitate in the reaction mixture was filtered, washed with a small amount of acetonitrile, and then excessively washed with saturated aqueous NaHCO₃ solution and water and was dried on the filter (crop 1, 28.0 g, purity 96%, major impurity: unreacted starting material, 5.7%, 215 nm). The filtrate was concentrated on rotary evaporator, the residue was diluted with 1:1 water-acetonitrile mixture (50 mL) and the solid was filtered, washed with water-acetonitrile mixture, aqueous NaHCO₃ solution and water. Drying on the filter afforded the second crop of the product (12.6 g, purity 90%, major impurity: 5′,7′-dibromo-4′-fluorospiro[cyclopropane-1,3′-indolin]-2′-one, 10%). Total yield of two crops 83%.

¹H NMR (CDCl₃, 300 MHz): δ 8.76 (br s, 1H), 7.35 (dd, J=6.8, 8.3 Hz, 1H), 6.69 (d, J=8.3 Hz, 1H), 1.95 (m, 1H), 1.73 (m, 1H).

LCMS (EI+), m/z: 256 (M+H). HPLC analytical method: Supelco Discovery® C8 column 5μ 4.6×50 cm column, 1 mL/min flow rate, gradient 10 to 90% over 9 min of MeCN-water containing 0.02% of TFA. Retention times: 5′-Bromo-4′-fluorospiro[cyclopropane-1,3′-indolin]-2′-one (5.18′), 4′-fluorospiro[cyclopropane-1,3′-indolin]-2′-one (4.30′), 7′-Bromo-4′-fluorospiro[cyclopropane-1,3′-indolin]-2′-one (4.99′), and 5′,7′-Bromo-4′-fluorospiro[cyclopropane-1,3′-indolin]-2′-one (5.83′).

### Example 5

### 5-(4′-fluoro-2′-oxospiro[cyclopropane-1,3′-indoline]-5′-yl)-1-methyl-1H-pyrrole-2-carbonitrile

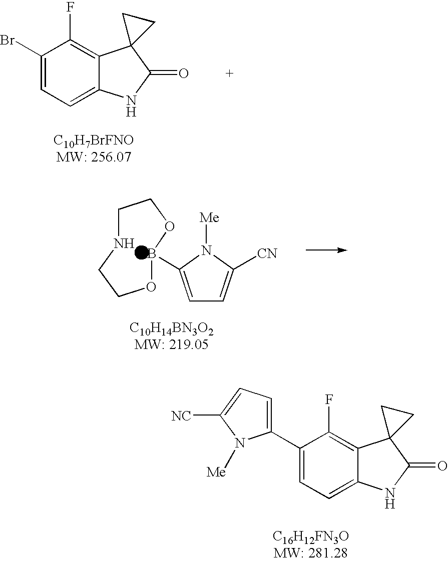

A 1-L round bottom flask equipped with a mechanical stirrer, a reflux condenser, a nitrogen inlet, and a thermocouple and set in a heating mantle, was purged with nitrogen and charged with 5′-bromo-4′-fluorospiro[cyclopropane-1,3′-indolin]-2′-one (28.0 g, 0.109 mol), NaHCO₃ (27.5 g, 0.327 mol, 3.0 eq), Pd₂(dba)₃ (1.13 g, 2.18 mmol, 0.02 eq Pd), and P(t-Bu)₃.HBF₄ (632 mg, 2.18 mmol, 0.02 eq). DME (350 mL) and water (80 mL) were added to the mixture, the heat was turned on and the mixture was heated to reflux. When the temperature reached 40° C., solid diethanolamine complex of 5-cyano-1-methyl-1H-pyrrol-2-ylboronic acid (28.7 g, 0.131 mol, 1.2 eq) was added to the mixture. After the reaction mixture reached reflux temperature (72° C.), HPLC showed completion of the reaction. The source of heat was removed and the reaction mixture was allowed to cool to room temperature.

The inorganic solid material and the precipitated palladium catalyst were removed by filtering the mixture through a paper filter. The filtrate was diluted with water (300 mL) which induced crystallization of the product. The precipitate was allowed to age for 3-4 hours at room temperature and then it was filtered and washed with 1:1 DME-water mixture, then water. The crude material, after drying overnight on the filter, resulted in 25.9 g (85%) of light-gray solid.

The crude product was dissolved in THF (300 mL) at room temperature. Activated carbon (7.0 g) was added to the THF solution, the slurry was stirred at room temperature for 3 hours and then filtered through a pad of Magnesol (about 2 cm high). The filtrate was concentrated on a rotary evaporator until the crystallized material formed a thick paste. The remaining THF was removed by co-distillation with ethanol (3×100 mL). The final slurry (about 100 mL) was chilled in ice, filtered, and the solid on the filter was washed with small amount of ice-cold ethanol. The filter cake was dried on the filter and then in a vacuum oven at 45° C. Yield 22.8 g (75%) as white crystalline solid

M.p. 222.5-3.8° C.

HPLC purity 99.0% (215 nm), LSI 0.3%.

¹H NMR (CDCl₃, 400 MHz): δ 9.50 (br s, 1H), 7.14 (dd, J=7.3, 8.1 Hz, 1H), 6.90 (d, J=8.0 Hz, 1H), 6.86 (d, J=4.0 Hz, 1H), 6.19 (d, J=4.0 Hz, 1H), 3.64 (2s, 3H), 1.98 (m, 2H), 1.77 (m, 2H).

¹³C NMR (CDCl₃, 100 MHz): δ 179.1, 154.3 (d, J=247 Hz), 143.8 (d, J=9.9 Hz), 133.6, 130.3 (d, J=2.7 Hz), 119.4, 117.4 (d, J=18.8 Hz), 114.1, 113.0 (d, J=14.7 Hz), 111.0, 106.6 (d, J=3.2 Hz), 105.5, 33.5, 27.0, 17.7.

MS (ES−), m/z: 280.1 (M−H).

For C₁₆H₁₂FN₃O, calc'd C, 68.32%; H, 4.30%; N, 14.94%; found C, 68.41%; H, 4.82%; N, 14.82%. Residual Pd content: 46 ppm.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

What is claimed is:

1. A process for dialkylating an indolinone compound of the following structure at the 3-position: 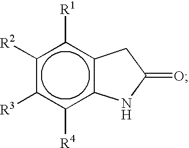
wherein:
R¹, R², R³, and R⁴ are, independently, selected from the group consisting of H, chlorine, fluorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂;
wherein said C₂ to C₆ alkynyl and substituted C₂ to C₆ alkynyl groups of R¹ to R⁴ comprise internal triple bonds; or
R¹ and R²; R² and R³; R³ and R⁴; R¹, R², and R³; or R², R³, and R⁴ are fused to form:
(i) a 3 to 15 membered saturated or unsaturated carbon-containing ring; or
(ii) a 3 to 15 membered heterocyclic ring containing in its backbone from 1 to 3 heteroatoms selected from the group consisting of O, S, and NR¹¹; and
R⁵ is selected from the group consisting of C₁ to C₆ alkyl and C₁ to C₆ substituted alkyl;
R¹¹ is absent, H, C₁ to C₆ alkyl, substituted C₁ to C₆ alkyl, aryl, or substituted aryl;
said process comprising (i) reacting said indolinone compound with at least 2 equivalents of a first base to form the di-anion of said indolinone compound; and (ii) reacting said di-anion with an alkylating agent in the presence of a second base.

2. The process according to claim 1, wherein said first base is an alkyl lithium, an alkali metal hydride, a Grignard reagent, an alkali metal alkyl amide, or an alkali metal disilazide.

3. The process according to claim 2, wherein said metal alkyl amide is lithium diisopropylamide.

4. The process according to claim 2, wherein said alkyl lithium is butyl lithium.

5. The process according to claim 2, comprising about 3 equivalents of alkyl lithium.

6. The process according to claim 1, wherein said second base comprises at least 1 equivalent of lithium diisopropylamide.

7. The process according to claim 1, wherein said first base is lithium diisopropylamide, which is added simultaneously with said second base comprising lithium diisopropylamide.

8. The process according to claim 1, wherein said first base is lithium diisopropylamide, which is added separately from said second base comprising lithium diisopropylamide.

9. The process according to claim 8, wherein said lithium diisopropylamide is added before said alkylating agent.

10. The process according to claim 1, wherein said indolinone compound reacts with said first base to form a dianion of said indolinone compound prior to said dialkylation.

11. The process according to claim 1, wherein said indolinone is 4-fluoroindolin-2-one.

12. The process according to claim 1, wherein the dialkylated indolinone compound is of the structure: 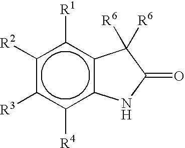
and said alkylating agent is R⁶X², wherein:
X² is halogen or OSO₂—R¹⁶; and
R¹⁶ is C₁ to C₁₀ alkyl, substituted C₁ to C₁₀ alkyl, aryl, or substituted aryl.

13. The process according to claim 1, wherein dialkylation of the 3-position of said indolinone is performed in the absence of N-alkylation.

14. The process according to claim 1, wherein the dialkylated indolinone is prepared at a greater than 90% yield.

15. A process for preparing a compound of the structure: 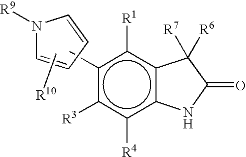
wherein:
R¹, R³, and R⁴ are, independently, selected from the group consisting of H, chlorine, fluorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂; or
R³ and R⁴ are fused to form:
(i) a 3 to 15 membered saturated or unsaturated carbon-containing ring; or
(ii) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl;
R⁶ and R⁷ are, independently, selected from the group consisting of C₁ to C₆ alkyl, substituted C₁ to C₆ alkyl, C₃ to C₁₄ cycloalkyl, substituted C₃ to C₁₄ cycloalkyl, aryl substituted aryl, heteroaryl, substituted heteroaryl, C₃ to C₆ alkenyl, substituted C₃ to C₆ alkenyl, C₃ to C₆ alkynyl, substituted C₃ to C₆ alkynyl, SR⁵, OR⁵, and N(R⁵)₂; or
R⁶ and R⁷ are fused to form a 3 to 8 membered saturated carbon-containing ring;
R⁹ is selected from the group consisting of C₁ to C₆ alkyl, substituted C₁ to C₆ alkyl, aryl, substituted aryl, heteroaryl, substituted heteroaryl, and COR^{A};
R^{A} is selected from the group consisting of H, C₁ to C₆ alkyl, substituted C₁ to C₆ alkyl, C₁ to C₆ alkoxy, substituted C¹ to C₆ alkoxy, C¹ to C₆ aminoalkyl, and substituted C₁ to C₆ aminoalkyl;
R¹⁰ is selected from the group consisting of H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A};
wherein said process comprises reacting a compound of the structure 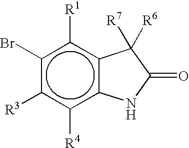
with a pyrrole compound of the structure: 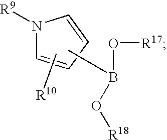
wherein:
R¹⁷ and R¹⁸ are, independently, H, C₁ to C₆ alkyl, or substituted C₁ to C₆ alkyl; or
R¹⁷ and R¹⁸ are fused to form:
(i) a saturated carbon-atom based 3 to 8 membered ring; or
(ii) a saturated carbon-atom based 3 to 8 membered ring containing one or more additional heteroatoms selected from the group consisting of O, S, and NR¹⁹;
R¹⁹ is H, C₁ to C₆ alkyl, or substituted C₁ to C₆ alkyl;
in the presence of a palladium catalyst comprising a phosphine ligand.

16. The process according to claim 15, wherein:
R¹⁷ and R¹⁸ are fused to form —(CR²⁰₂)—(CH₂)ₙ—(CR²⁰₂)—;
n is 0 to 6; and
R²⁰ is, independently, H or C₁ to C₆ alkyl

17. The process according to claim 15, wherein:
R¹⁷ and R¹⁸ are fused to form —(CH₂)ₘ—(NR¹⁹)—(CH₂)_{q}—;
m is 1 to 6; and
q is 1 to 6.

18. The process according to claim 15, wherein said pyrrole compound is:

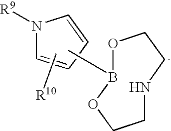

19. A process for preparing a compound of the structure: 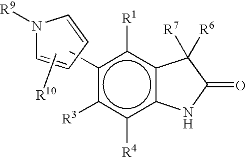
wherein:
R¹, R³, and R⁴ are, independently, selected from the group consisting of H, chlorine, fluorine, C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, CF₃, SR⁵, OR⁵, N(R⁵)₂, and SO₂N(R⁵)₂; or
R³ and R⁴ are fused to form:
(a) a 3 to 15 membered saturated or unsaturated carbon-containing ring; or
(b) a 3 to 15 membered heterocyclic ring containing in its backbone from 1 to 3 heteroatoms selected from the group consisting of O, S, and NR¹¹; and
R⁵ is selected from the group consisting of C₁ to C₆ alkyl and C₁ to C₆ substituted alkyl;
R⁶ and R⁷ are the same and are selected from the group consisting of C₁ to C₆ alkyl, substituted C₁ to C₆ alkyl, C₃ to C₁₄ cycloalkyl, and substituted C₃ to C₁₄ cycloalkyl; or
R⁶ and R⁷ are fused to form a 3 to 8 membered saturated carbon-containing ring;
R⁹ is selected from the group consisting of C₁ to C₆ alkyl, substituted C₁ to C₆ alkyl, aryl, substituted aryl, heteroaryl, substituted heteroaryl, and COR^{A};
R^{A} is selected from the group consisting of H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl;
R¹⁰ is selected from the group consisting of H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A};
said process comprising:
(i) protecting the amine group of a compound of the structure: 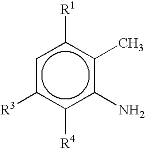
(ii) lithiating the product of step (i);
(iii) reacting the product of step (ii) with CO₂ or Y¹C(O)X⁵;
wherein, X⁵ and Y¹ are independently chlorine, bromine, C₁ to C₆ alkoxy, or substituted C₁ to C₆ alkoxy;
(iv) deprotecting the product of step (iii);
(v) cycloamidating the product of step (iv) to form a compound of the structure: 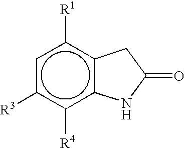
(vi) dialkylating the product of step (v) in the presence of at least 2 equivalents of a first base, at least 1 equivalent of lithium diisopropylamide, and at least 2 equivalents of an alkylating agent to form a compound of the structure: 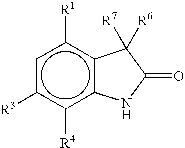
(vii) brominating the product of step (vi) to form a compound of the structure: 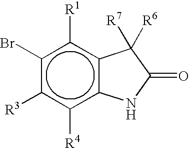
(viii) reacting the product of step (vii) with a compound of the structure:

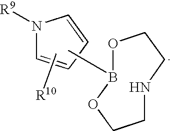

20. A process for preparing 5-(4′-fluoro-2′-oxospiro[cyclopropane-1,3′-indoline]-5′-yl)-1-methyl-1H-pyrrole-2-carbonitrile, comprising:
(i) BOC protecting a compound of the structure: 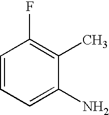
to form a compound of the structure: 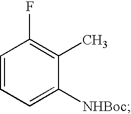
(ii) lithiating the product of step (i) to form a compound of the structure: 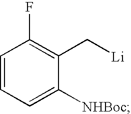
(iii) reacting the product of step (ii) with carbon dioxide to form a compound of the structure: 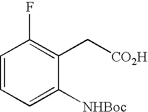
(iv) deprotecting the product of step (iii);
(v) cycloamidating the product of step (iv) to form a compound of the structure: 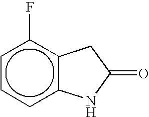
(vi) dialkylating the compound of step (v) using about 3 to about 4 equivalents of lithium diisopropylamide and at least 2 equivalents of 1,2-dibromoethane to form a compound of the structure: 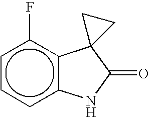
(vii) brominating the product of step (vi) to form a compound of the structure: 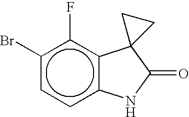
(viii) reacting the product of step (vii) with a compound of the structure:

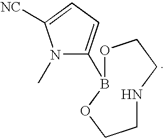

21. A method of preparing 4′-Fluorospiro[cyclopropane-1,3′-indolin]-2′-one, comprising:
(i) reacting 4-fluoroindolin-2-one and lithium diisopropylamide; and
(ii) adding 1,2-dibromoethane to the product of step (i).

22. The method according to claim 21, comprising about 3 equivalents of 1,2-dibromoethane.
